(19) United States
(12) Patent Application Publication
(10) Pub. No.: US 2007/0237720 A1
(43) Pub. Date: Oct. 11, 2007

(54) Embolic prosthesis for treatment of vascular aneurysm

(76) Inventors: Orlando Padilla, Laguna Niguel, CA (US); Andrew Morris, San Diego, CA (US); John Nguyen, San Diego, CA (US); Eric V. Schmid, San Diego, CA (US); Donald K. Brandom, Davis, CA (US); James E. McGrath, Blacksburg, VA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/399,136

(22) Filed: Apr. 6, 2006

(51) Int. Cl. A61K 49/04; A61M 31/00

(52) U.S. Cl. 424/9.41; 604/500

(57) ABSTRACT

The invention relates to an implantable embolic medical device comprising a non-erodible, erodible or biodegradable material. The device preferably comprises one or more longitudinal filament members of varying cross sectional shapes which may or may not be coiled to suit a particular clinical need. The embolic device is placed through lumens and cavities to reach areas in the body which require embolism to achieve a particular clinical objective.

### BACKGROUND OF THE INVENTION

1. Field of the Invention

The invention relates generally to medical systems and methods for forming an occlusion in a mammalian body. More particularly, the invention relates to systems and methods for the treatment of conditions for which a restricted blood supply may be therapeutic, such as vascular aneurysms, with an implantable embolic device that can be resorbable, non-resorbable, erodible or non-erodible.

2. Description of the Related Art

Like many parts of the body, the brain is composed of living cells that requires a blood supply to provide oxygen and nutrients. A hemorrhage in a blood vessel in the brain or in the space closely surrounding the brain is a common cause of strokes. Hemorrhage refers to bleeding into the brain, usually because of a problem with a blood vessel. The problem is often an aneurysm.

An aneurysm is an abnormal outward bulging of a blood vessel wall. If the aneurysm ruptures, a hemorrhage occurs. This can compress and irritate the surrounding blood vessels, thereby resulting in a reduced supply of oxygen and nutrients to the cells, and hence possibly causing a stroke.

Aneurysms can be treated from outside the blood vessel using surgical techniques or from inside the blood vessel using endovascular techniques. Endovascular treatment of an aneurysm is typically performed using a catheter to deliver an embolic coil for treating the aneurysm. Visualization equipment may be used to view the progress during the procedure.

There has been progress in endovascular surgery. But there are still unresolved issues regarding the use, safety and efficacy relating to the treatment of cerebral aneurysms using conventional embolic coils and surgery techniques. These include surgical and post-surgical risks and complications.

Complications include incomplete occlusion of the aneurysm, rupture or re-rupture of the aneurysm during placement of the coils, thromboembolism, vasospasm, need for additional patient interventions at a later date, and re-bleeding at a future date. (Thromboembolism is a blood clot that forms and then breaks off and travels through the bloodstream to another part of the body. Cerebral vasospasm is narrowing of arteries in the brain.) Thus, conventional treatments of cerebral aneurysms have a success rate that is at an unsatisfactory level and improvements are both desired and needed.

### SUMMARY OF THE INVENTION

Advantageously, embodiments of the invention substantially overcome or mitigate some or all of the above-mentioned disadvantages by providing an implantable embolic medical device comprising a non-erodible, erodible or biodegradable material. The device preferably comprises one or more longitudinal filament members of varying cross sectional shapes which may or may not be coiled to suit a particular clinical need. The embolic device is placed through lumens and cavities to reach areas in the body which require embolism to achieve a particular clinical objective.

In some embodiments, the filament members comprise radiopaque or non-radiopaque polymers. In some embodiments, the filament members comprise resorbable or non-resorbable polymers. In some embodiments the filaments comprise radiopaque or nonradiopaque metals. In some embodiments, the filament members comprise erodible or non-erodible metals. In some embodiments, the filament members comprise shape memory metals such as, but not limited to, Nitinol and spring steel. Any combination of these embodiments may be efficaciously utilized, as needed or desired.

In preferred embodiments of the embolic filaments, the filament members may be made from polymers selected from the group consisting of those polymers described in U.S. Pat. No. 6,475,477, and co-pending U.S. application Ser. Nos. 10/952,202, 10/952,274, 11/176,638, 11/200,656 and 11/335,771; all of which are incorporated herein in their entirety by reference thereto.

In one preferred embodiment, the filament members may comprise a polymer described in Ser. No. 10/952,202 as a polymer comprising one or more units described by Formula I:

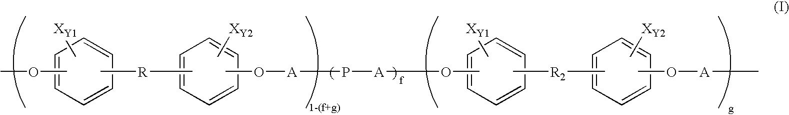

wherein each X is independently I or Br, Y1 and Y2 for each diphenol unit are independently between 0 and 4, inclusive, and Y1+Y2 for each diphenol unit is between 1 and 8, inclusive.

wherein each R and R2 are independently an alkyl, aryl or alkylaryl group containing up to 18 carbon atoms and from 0 to 8 heteroatoms selected from O and N and R2 further comprises a pendant free carboxylic acid group;

wherein A is either:

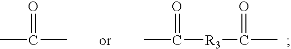

wherein R3 is a saturated or unsaturated, substituted or unsubstituted alkyl, aryl, or alkylaryl group containing up to about 18 carbon atoms and 0 to 8 heteroatoms selected from O and N;

wherein P is a poly(C1-C4 alkylene glycol) unit; f is from 0 to less than 1; g is from 0 to 1, inclusive; and f+g ranges from 0 to about 1, inclusive.

Preferably, iodine and bromine are both present as ring substituents. Further, all X groups are preferably ortho-directed. Y1 and Y2 may independently be 2 or less, and Y1+Y2=1, 2, 3 or 4. In another variation, Y1+Y2=2 or 3. All X groups are preferably iodine.

In another variation to the present invention, the weight fraction of the poly(C1-C4 alkylene glycol) unit is less than about 75 wt %. In a preferred variation, the weight fraction of the poly(C1-C4 alkylene glycol) unit is less than about 50 wt %. More preferably, the poly(C1-C4 alkylene glycol) is poly(ethylene glycol) with a weight fraction of less than about 40 wt %. Most preferably, the weight fraction of the poly(ethylene glycol) unit is between about 1 and 25 wt %. P may independently be C1 up to C4 or copolymers of C1-C4.

In another variation to the present invention, f may vary between about 0 and 0.5, inclusive. Preferably, f is less than about 0.25. More preferably, f is less than about 0.1. More preferably yet, f varies from about 0.001 to about 0.08. Most preferably, f varies between about 0.025 and about 0.035.

In another variation to the present invention, g is greater than 0 and typically varies between greater than 0 and about 0.5, inclusive. Preferably, g is greater than about 0.1 to about 0.35. More preferably, g is from about 0.2 to about 0.3. More preferably yet, g varies between about 0.01 and about 0.25. Most preferably, g is between about 0.05 and about 0.15.

In another variation to the present invention, R2 further comprises a pendant carboxylic acid group. Preferably, both R and R2 comprise a pendant COOR1 group; wherein for R, the subgroup R1 is independently an alkyl group ranging from 1 to about 18 carbon atoms containing from 0 to 5 heteroatoms selected from O and N; and wherein for R2, the subgroup R1 is a hydrogen atom. In another preferred embodiment, each R and R2 independently has the structure:

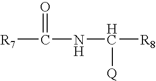

wherein R7 is selected from the group consisting of —CH═CH—, —CHJ1-CHJ2- and (—CH2-)a; wherein R8 is selected from the group consisting of —CH═CH—, —CHJ1CHJ2- and (—CH2-)n; wherein a and n are independently between 0 and 8 inclusive; and J1 and J2 are independently Br or I; and wherein, for each R2, Q comprises a free carboxylic acid group, and for each R, Q is independently selected from the group consisting of hydrogen and carboxylic acid esters and amides, wherein said esters and amides are selected from the group consisting of esters and amides of alkyl and alkylaryl groups containing up to 18 carbon atoms and esters and amides of biologically active compounds.

In a preferred variation to the present invention, each R and R2 independently has the structure:

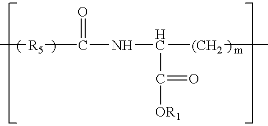

wherein R5 is an alkyl group containing up to 18 carbon atoms and from 0 to 5 heteroatoms selected from O and N; and wherein m is an integer from 1 to 8 inclusive; and wherein, for each R2, R1 is hydrogen, and, for each R, R1 is independently an alkyl group ranging from 1 to about 18 carbon atoms containing from 0 to 5 heteroatoms selected from O and N.

In a more preferred variation to the present invention, each R and R2 independently has the structure:

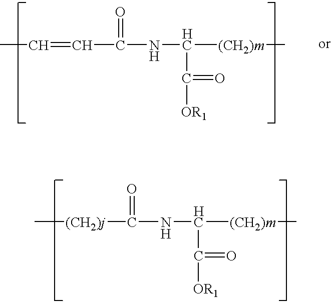

wherein j and m are independently an integer from 1 to 8, inclusive, and wherein, for each R2, R1 is hydrogen, and, for each R, R1 is independently an alkyl group ranging from 1 to about 18 carbon atoms containing from 0 to 5 heteroatoms selected from 0 and N.

Preferably, each R1 subgroup for R is independently an alkyl group ranging from 1 to about 18 carbon atoms and containing from 0 to 5 heteroatoms selected from O and N. More preferably, each R1 subgroup for R is independently either ethyl or butyl.

In another variation to the present invention, A is a —C(═O)— group. Alternatively, A may be:

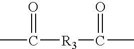

wherein R3 is a C4-C12 alkyl, C8-C14 aryl, or C8-C14 alkylaryl. Preferably, R3 is selected so that A is a moiety of a dicarboxylic acid that is a naturally occurring metabolite. More preferably, R3 is selected from the group consisting of —CH2-C(═O)—, —CH2-CH2-C(═O)—, —CH═CH— and (—CH2-)z; and wherein z is an integer from 0 to 8, inclusive. More preferably, z is an integer from 1 to 8, inclusive.

In one preferred embodiment, the filament members may comprise a polymer described in Ser. No. 10/952,274 as having one or more units described by Formula II:

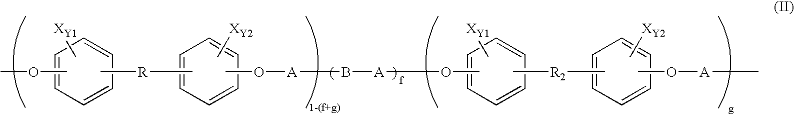

wherein X═I or Br; Y1 and Y2 can independently=0, 1, 2, 3 or 4;

wherein f is between 0 and less than 1; g is between 0 and 1, inclusive; and f+g is between 0 and 1, inclusive;

wherein A is either:

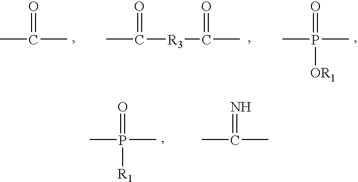

wherein R₁ is independently an H or an alkyl group ranging from 1 to about 18 carbon atoms containing from 0 to 5 heteroatoms selected from O and N;

wherein R₃ is a saturated or unsaturated, substituted or unsubstituted alkyl, aryl, or alkylaryl group containing up to about 18 carbon atoms and 0 to 8 heteroatoms selected from O and N;

wherein B is an aliphatic linear or branched diol or a poly(alkylene glycol) unit; and

wherein R and R₂ may be independently selected from:

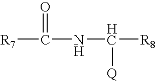

wherein R₇ is selected from the group consisting of —CH═CH—, —CHJ₁-CHJ₂- and (—CH₂-)a; wherein R₈ is selected from the group consisting of —CH═CH—; —CHJ₁-CHJ₂- and (CH₂-)n; wherein a and n are independently between 0 and 8 inclusive; J₁ and J₂ are independently Br or I; and, for R₂, Q comprises a free carboxylic acid group, and, for R, Q is selected from the group consisting of hydrogen and carboxylic acid esters and amides, wherein said esters and amides are selected from the group consisting of esters and amides of alkyl and alkylaryl groups containing up to 18 carbon atoms and esters and amides of biologically pharmaceutically active compounds.

In a variation to this embodiment of Formula II, R and R₂ may be selected from the groups:

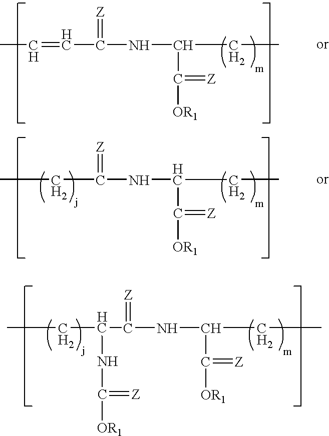

wherein R₁ in each R₂ is independently an alkyl group ranging from 1 to about 18 carbon atoms containing from 0 to 5 heteroatoms selected from O and N and R₁ in each R is H;

wherein j and m are independently integers from 1 to 8 inclusive; and

wherein Z is independently either O or S.

In another preferred embodiment, the polymer may comprise one or more units described by Formula III:

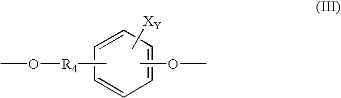

wherein X for each polymer unit is independently Br or I, Y is between 1 and 4, inclusive and R₄ is an alkyl, aryl or alkylaryl group with up to 18 carbon atoms and from 0 to 8 heteroatoms selected from O and N.

In variations to the polymer of Formula III, all X groups may be ortho-directed an Y may be 1 or 2. In another variation, R₄ is an alkyl group.

In another variation, R₄ has the structure:

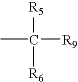

wherein R₉ for each unit is independently an alkyl, aryl or alkylaryl group containing up to 18 carbon atoms and from 0 to 8 heteroatoms selected from O and N; and R₅ and R₆ are each independently selected from hydrogen and alkyl groups having up to 18 and from 0 to 8 heteroatoms selected from O and N.

In another variation to R₄ in Formula III, R₉ for at least one unit comprises a pendent COOR₁ group, wherein, for each unit in which it is present, the subgroup R₁ is independently a hydrogen or an alkyl group ranging from 1 to about 18 carbon atoms containing from 0 to 5 heteroatoms selected from O and N.

In another variation to R₄ in Formula III, R₉ independently has the structure:

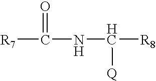

wherein R₇ is selected from the group consisting of —CH═CH—, —CHJ₁-CHJ₂- and (—CH₂-)a, wherein R₈ is selected from the group consisting of —CH═CH—, —CHJ₁-CHJ₂- and (—CH₂-)n, wherein a and n are independently between 0 and 8 inclusive; and J₁ and J₂ are independently Br or I; and Q is selected from the group consisting of hydrogen, a free carboxylic acid group, and carboxylic acid esters and amides, wherein said esters and amides are selected from the group consisting of esters and amides of alkyl and alkylaryl groups containing up to 18 carbon atoms and esters and amides of biologically and pharmaceutically active compounds.

In another variation to R₄ in Formula III, R₉ independently has the structure:

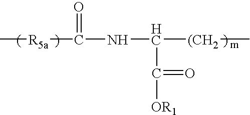

wherein R₅ₐ is an alkyl group containing up to 18 carbon atoms and from 0 to 5 heteroatoms selected from O and N; and wherein m is an integer from 1 to 8 inclusive; and R₁ is independently a hydrogen or an alkyl group ranging from 1 to about 18 carbon atoms containing from 0 to 5 heteroatoms selected from O and N.

In another variation to R₄ in Formula III, R₉ independently has the structure:

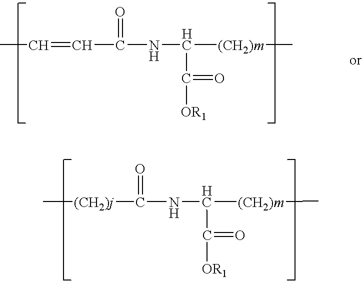

wherein j and m are independently an integer from 1 to 8, inclusive, and R₁ is independently a hydrogen or an alkyl group ranging from 1 to about 18 carbon atoms containing from 0 to 5 heteroatoms selected from O and N.

In some embodiments, the polymer may be copolymerized with a poly(C₁-C₄ alkyl glycol). Preferably, the poly(C₁-C₄ alkylene glycol) is present in a weight fraction of less than about 75 wt %. More preferably, the poly(alkylene glycol) is poly(ethylene glycol).

In another variation to the polymers disclosed herein, between about 0.01 and about 0.99 percent of said polymer units comprise a pendant —COOH group.

In another variation to Formula III, R₄ may be an aryl or alkylaryl group. Preferably, the R₄ aryl or alkylaryl group is selected so that the polymer units are diphenols.

In another preferred embodiment, the polymer may comprise one or more units described by Formula IV:

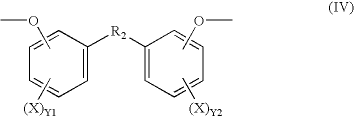

wherein X for each polymer unit is independently Br or I, Y1 and Y2 are each independently between 0 and 4, inclusive, Y1+Y2 for each unit is independently between 1 and 8, inclusive, and R₂ for each polymer unit is independently an alkyl, aryl or alkylaryl group containing up to 18 carbon atoms and from 0 to 8 heteroatoms selected from O and N.

In preferred variations to Formula IV, all X groups are ortho-directed. Preferably, Y1 and Y2 are independently 2 or less, and Y1+Y2=1, 2, 3 or 4.

In another variation to Formula IV, R₂ for at least one unit may comprise a pendent COOR₁ group, wherein, for each unit in which the COOR₁ group is present, the subgroup R₁ independently a hydrogen or an alkyl group ranging from 1 to about 18 carbon atoms containing from 0 to 5 heteroatoms selected from O and N.

In another variation to Formula IV, R₂ independently has the structure:

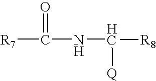

wherein R₇ is selected from the group consisting of —CH═CH—, —CHJ₁-CHJ₂- and (—CH₂-)a, wherein R₈ is selected from the group consisting of —CH═CH—, —CHJ₁-CHJ₂- and (—CH₂-)n, wherein a and n are independently between 0 and 8 inclusive; and J₁ and J₂ are independently Br or I; and Q is selected from the group consisting of hydrogen, a free carboxylic acid group, and carboxylic acid esters and amides, wherein said esters and amides are selected from the group consisting of esters and amides of alkyl and alkylaryl groups containing up to 18 carbon atoms and esters and amides of biologically and pharmaceutically active compounds.

In another variation to Formula IV, R₂ independently has the structure:

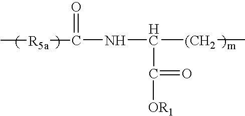

wherein R₅ₐ is an alkyl group containing up to 18 carbon atoms and from 0 to 5 heteroatoms selected from O and N; and wherein m is an integer from 1 to 8 inclusive; and R₁ is independently a hydrogen or an alkyl group ranging from 1 to about 18 carbon atoms containing from 0 to 5 heteroatoms selected from O and N.

In another variation to Formula IV, R₂ independently has the structure:

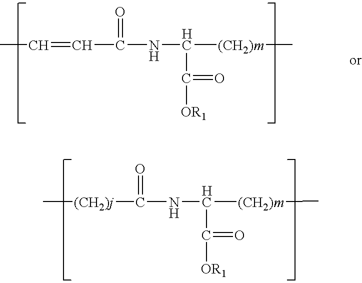

wherein j and m are independently an integer from 1 to 8, inclusive, and R₁ is independently a hydrogen or an alkyl group ranging from 1 to about 18 carbon atoms containing from 0 to 5 heteroatoms selected from O and N.

In a preferred variation to Formula IV, between about 0.01 and about 0.99 percent of the polymer units comprise a pendant COOH group. Preferably, the polymer is copolymerized with up to 75 wt % of a poly(C₁-C₄ alkylene glycol). More preferably, the poly(C₁-C₄ alkylene glycol) is poly(ethylene glycol).

In another preferred embodiment, the polymer may comprise one or more units described by Formula V:

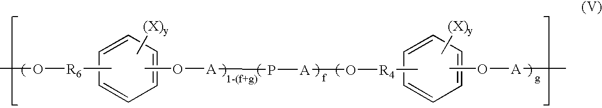

wherein each X is independently iodine or bromine; each y is independently between 0 and 4, inclusive, wherein a total number of ring-substituted iodine and bromine is between 1 and 8, inclusive; each R₄ and R₆ are independently an alkyl, aryl or alkylaryl group containing up to 18 carbon atoms and from 0 to 8 heteroatoms selected from O and N, and R₄ further includes a pendant carboxylic acid group;

wherein A is either:

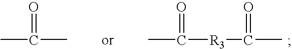

wherein R₃ is a saturated or unsaturated, substituted or unsubstituted alkyl, aryl, or alkylaryl group containing up to about 18 carbon atoms and 0 to 5 heteroatoms selected from the group consisting of O and N;

P is a poly(C₁-C₄ alkylene glycol) unit present in a weight fraction of less than about 75 wt %;

f is from greater than 0 to less than 1; g is between 0 and 1, inclusive; and f+g is between 0 and 1, inclusive.

Preferably, P is a poly(ethylene glycol) unit.

In preferred variations to Formula V, each R₄ and R₆ of said polymer contains a pendant —COOR₁ group, wherein for each R₆, each subgroup R₁ is independently an alkyl group ranging from 1 to about 18 carbon atoms containing from 0 to 5 heteroatoms selected from the group consisting of O and N, and, for each R₄, each subgroup R₁ is a hydrogen atom.

In other preferred variations to Formula V, each R₄ and R₆ of said polymer are:

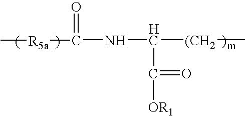

wherein R₅ₐ is an alkyl group containing up to 18 carbon atoms and from 0 to 5 heteroatoms selected from O and N; and wherein m is an integer from 1 to 8 inclusive; and for each R₆, each subgroup R₁ is independently an alkyl group ranging from 1 to about 18 carbon atoms containing from 0 to 5 heteroatoms selected from O and N, and, for each R₄, each subgroup R₁ is a hydrogen atom.

In other preferred variations to Formula V, each R₁ subgroup for R₆ of said polymer is either ethyl or butyl.

In other preferred variations to Formula V, A is a —C(═O)— group. Alternatively, A may be:

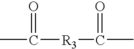

wherein R₃ is C₄-C₁₂ alkyl, C₈-C₁₄ aryl, or C₈-C₁₄ alkylaryl.

In other preferred variations to Formula V, R₃ is selected so that A is a moiety of a dicarboxylic acid that is a naturally occurring metabolite.

In other preferred variations to Formula V, R₃ is a moiety selected from the group consisting of —CH₂—C(═O)—, —CH₂—CH₂—C(═O)—, —CH═CH— and (—CH₂-)z, wherein z is an integer from 1 to 8, inclusive.

In other preferred variations to Formula V, all X groups are ortho-directed and y is 2 or 3.

In other preferred variations to Formula V, every X group is iodine.

In other preferred variations to Formula V, f is greater than 0.1 to about 0.3.

In other preferred variations to Formula V, g is greater than 0.1 to about 0.35.

In one preferred embodiment, the filament members may comprise an inherently radiopaque side chain crystallizable polymer, comprising a main chain, a plurality of crystallizable side chains, and a plurality of heavy atoms attached to the polymer, the heavy atoms being present in an amount that is effective to render the polymer radiopaque. A polymer that comprises a recurring unit of the formula (VI) is an example of such an inherently radiopaque side chain crystallizable polymer:

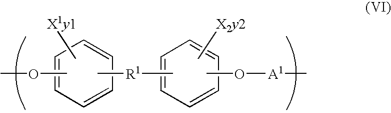

In formula (VI), X¹ and X² are each independently selected from the group consisting of Br and I; y¹ and y² are each independently zero or an integer in the range of 1 to 4; and A¹ is selected from the group consisting of

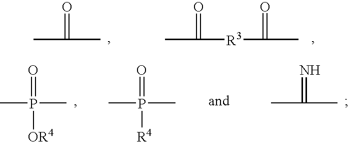

R³ is selected from the group consisting of C₁-C₃₀ alkyl, C₁-C₃₀ heteroalkyl, C₅-C₃₀ aryl, C₆-C₃₀ alkylaryl, and C₂-C₃₀ heteroaryl; R⁴ selected from the group consisting of H, C₁-C₃₀ alkyl, and C₁-C₃₀ heteroalkyl; R¹ is

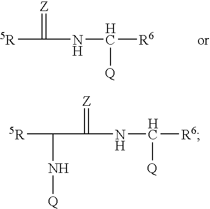

R⁵ and R⁶ are each independently selected from the group consisting of —CH═CH—, —CHJ¹-CHJ²-, and —CH₂)ₐ—; a is zero or an integer in the range of 1 to 8; J¹ and J² are each independently selected from the group consisting of Br and I; and Z is an O or an S; and q is a crystallizable group comprising from about 6 to about 30 carbon atoms, preferably from about 20 to about 30 carbon atoms. In an embodiment, Q is:

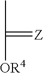

Polymers of the formula (VI) may be prepared by modifying the general methods described in U.S. patent application Ser. No. 11/200,656, to select the appropriate side chain length, side chain spacing and halogen content.

It will be recognized that Q and/or R⁴ may comprise crystallizable side chains, that each of X, J¹ and J² is a heavy atom, and that y may be adjusted so that the number of heavy atoms in the polymer is sufficient to render the polymer radiopaque. Q and R⁴ may each independently comprise units selected from the group consisting of —(CH₂)ₙ₁— and —((CH₂)ₘ₁—O—)ₙ₁; where m1 and n1 are each independently selected so that Q and/or R⁴ each independently contain from about 1 to about 30 carbon atoms, preferably from about 6 to about 30 carbon atoms, and more preferably from about 20 to 30 carbon atoms. Moreover, Q and R⁴ may include other functional groups such as ester and amide, and/or heavy atoms such as iodine and bromine. Non-limiting examples of Q and R⁴ thus include —Cₙ₁H₂ₙ₊₁, —CO₂—Cₙ₁H₂ₙ₁₊₁, —CONH—Cₙ₁H₂ₙ₁₊₁, —(CH₂)ₙ₁—Br, —(CH₂)ₙ₁—I, —CO₂—(CH₂)ₙ₁—BR, —CO₂—(CH₂)ₙ₁—I, —CONH—CO₂—(CH₂)ₙ₁—Br, and —CONH—CO₂—(CH₂)ₙ₁—I. In an embodiment, R⁵ is —CH═CH— or —(CH₂)ₐ—; R⁶ is —(CH₂)ₐ—; and Q is an ester group comprising from about 10 to about 30 carbon atoms.

It will be understood that a polymer that comprises a recurring unit of the formula (I) may be a copolymer, e.g., a polymer of the formula (I) that further comprises recurring —R²-A²- units, where R² is selected from the group consisting of —(CH₂)ₙ₂— and —((CH₂)ₘ₂—O—)ₙ₂; where m2 and n2 are each independently selected so that R² contains from about 1 to about 30 carbon atoms; and where A² is defined in the same manner as A¹ above. Thus, an embodiment provides a polymer comprising recurring units of the formula (VIa):

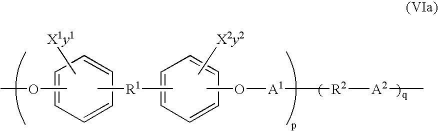

In formula (VIa), X¹, X², y¹, y², R¹ and A¹ are defined as described above for formula (VI); p and q may each be independently varied over a broad range to provide a polymer having the desired properties, e.g., melting point, radiopacity, and viscosity, using routine experimentation. In an embodiment, p and q are each independently an integer in the range of 1 to about 10,000. It will be appreciated that the formula (VI) units and —(R²-A²)- units in a polymer comprising recurring units of the formula (VIa) may be arranged in various ways, e.g., in the form of a block copolymer, random copolymer, alternating copolymer, etc.

Another embodiment of an inherently radiopaque side chain crystallizable polymer (e.g., a polymer comprising a main chain, a plurality of crystallizable side chains, and a plurality of heavy atoms attached to the polymer, the heavy atoms being present in an amount that is effective to render the polymer radiopaque), comprises a recurring unit of the formula (VII):

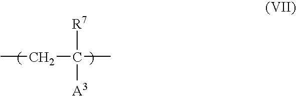

In formula (VII), R⁷ is H or CH₃; A³ is a chemical group having a molecular weight of about 500 or less; and A³ bears at least one of the heavy atoms attached to the polymer. Non-limiting examples of A³ include metal carboxylate (e.g., —CO₂Cs), metal sulfonate (e.g., —SO₄Ba), halogenated alkyl ester (e.g., —CO₂—(CH₂)_{b}—Br), halogenated alkyl amide (e.g., —CONH—(CH₂)_{b}—Br), and halogenated aromatic (e.g., —C₆H₄—I), where b is an integer in the range of about 1 to about 4. In an embodiment, A³ comprises an aromatic group bearing at least one halogen atom selected from the group consisting of bromine and iodine. In another embodiment, A³ comprises a chemical group of the formula -L₁—(CH₂)ₙ₃-L₂-Ar¹, wherein L¹ and L₂ each independently represent a nullity (i.e., are not present), ester, ether or amide group; n3 is zero or an integer in the range of about 1 to about 30; and Ar¹ comprises a halogenated aromatic group containing from about 2 to about 20 carbon atoms. Inherently radiopaque side chain crystallizable polymers that comprise a recurring unit of the formula (VII) may be formed by polymerization of the corresponding monomers or by post-reaction of appropriate polymeric precursors. Inherently radiopaque side chain crystallizable polymers that comprise a recurring unit of the formula (VII) may be copolymers that include additional recurring units.

Side chain A³ groups in an inherently radiopaque side chain crystallizable polymer comprising a recurring unit of the formula (VII) may be crystallizable and/or the inherently radiopaque side chain crystallizable polymer comprising a recurring unit of the formula (VII) may further comprise a second recurring unit that comprises a crystallizable side chain. Examples of suitable second recurring units having crystallizable side chains include the following: poly(1-alkene)s, poly(alkyl acrylate)s, poly(alkyl methacrylate)s, poly(alkyl vinyl ether)s, and poly(alkyl styrene)s. The alkyl groups of the foregoing exemplary second recurring units preferably contain more than 6 carbon atoms, and more preferably contain from about 6 to about 30 carbon atoms. For example, in an embodiment, the second recurring unit is of the formula (VIII):

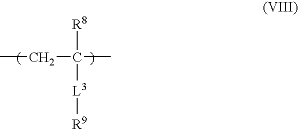

In formula (VIII), R⁸ is H or CH₃; L³ is an ester or amide linkage; and R⁹ comprises a C₆ to C₃₀ hydrocarbon group. Inherently radiopaque side chain crystallizable polymers comprising a recurring unit of the formula (VII) and a second recurring unit (such as a recurring unit of the formula (VIII)) may be formed by copolymerization of the corresponding monomers and/or by post reaction of appropriate polymeric precursors.

Another embodiment of an inherently radiopaque side chain crystallizable polymer (e.g., a polymer comprising a main chain, a plurality of crystallizable side chains, and a plurality of heavy atoms attached to the polymer, the heavy atoms being present in an amount that is effective to render the polymer radiopaque) comprises a recurring unit of the formula (IX), where A³ is defined above:

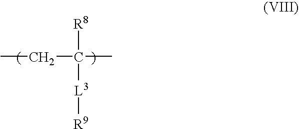

In formula (IX), A⁴ represents H or a group containing from about 1 to about 30 carbons, e.g., a C₁-C₃₀ hydrocarbon. Side chain A³ and/or A⁴ groups in an inherently radiopaque side chain crystallizable polymer may comprise a recurring unit of the formula (IX) and may further comprise a second recurring unit that comprises a crystallizable side chain. For example, in an embodiment, the second recurring unit is of the formula (X), where R¹⁰ comprises a C₆ to C₃₀ hydrocarbon group and R¹¹ represents H or a group containing from about 1 to about 30 carbons, e.g., a C₁-C₃₀ hydrocarbon:

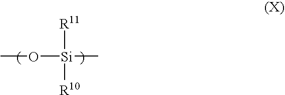

In one preferred embodiment, the filament members may comprise a polymer described in Ser. No. 11/335,771, comprising a recurring unit of the formula (XI):

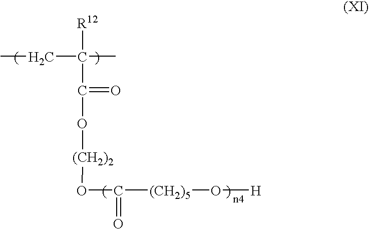

wherein R¹² is H or CH₃ and n4 is an integer in the range of about 1 to about 1,000. In preferred embodiments, the polymer comprising a recurring unit of the formula (XI) is biocompatible.

In one preferred embodiment, the filament members may comprise a polymer described in Ser. No. 11/200,656 as an inherently radiopaque, biocompatible, bioresorbable polymer, wherein the polymer comprises one or more recurring units of the Formula (XII):

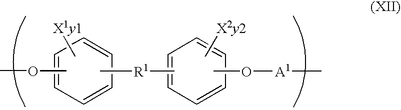

wherein:

X¹ and X² are each independently selected from the group consisting of Br and I;

y1 and y2 are each independently zero or an integer in the range of 1 to 4, with the proviso that the sum of y1 and y2 is at least one;

R¹ is

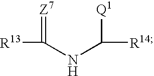

R¹³ and R¹⁴ are each independently selected from the group consisting of —CH═CH—, —(CH₂)_{c}—, —(CHJ¹)-, —CHJ², —CHJ³-, —CH═CH—(CHJ¹)-, and —(CH₂)_{c}—(CHJ¹)-;

c is zero or an integer in the range of 1 to 8;

J¹, J² and J³ are each independently selected from the group consisting of H, Br, I, —NH-Q² and —C(═Z⁸)-OQ³;

Q¹, Q² and Q³ are each independently H or a non-crystallizable group comprising from about 1 to about 30 carbons;

Z⁷ and Z⁸ are each independently O or S;

A¹ is selected from the group consisting of

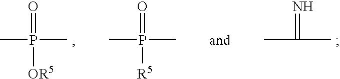

R⁵ is selected from the group consisting of H, C₁-C₃₀ alkyl, and C₁-C₃₀ heteroalkyl. In a preferred embodiment, X¹, X², y1 and y2 are selected so that X¹ and X² are present in an amount that is effective to render the polymer radiopaque.

In an embodiment of a polymer comprising a recurring unit of the Formula (XII), R¹ in Formula (XII) is:

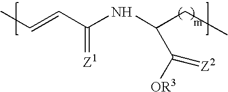

wherein R³ is H or a non-crystallizable C₁ to C₂₉ hydrocarbon;

Z¹ and Z² are each independently O or S; and

m is an integer in the range of 1 to 8.

In another embodiment of a polymer comprising a recurring unit of the Formula (XII), R¹ in Formula (XII) is:

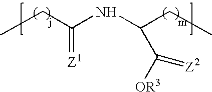

wherein R³ is H or a non-crystallizable C₁ to C₂₉ hydrocarbon;

Z¹ and Z² are each independently O or S; and

j and m are each independently an integer in the range of 1 to 8.

In another embodiment of a polymer comprising a recurring unit of the Formula (XII), R¹ in Formula (XII) is:

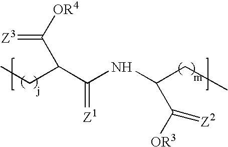

wherein R³ and R⁴ are each independently H or a non-crystallizable C₁ to C₂₉ hydrocarbon;

Z¹, Z² and Z³ are each independently O or S; and

j and m are each independently an integer in the range of 1 to 8.

Another embodiment provides a filament that comprises an inherently radiopaque, biocompatible, bioresorbable polymer, wherein the polymer comprises one or more recurring units of the Formula (XII) as described above.

Another embodiment provides an inherently radiopaque, biocompatible, bioresorbable polymer, wherein the polymer comprises one or more recurring units of the Formula (XII) as defined above, and further comprises one or more recurring units of the Formula (XIII):

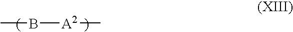

wherein:

B is —O—(CHR⁶)ₚ—O)_{q}—;

R⁶ is H or C¹ to C₃ alkyl;

p and q are each individually an integer in the range of about 1 to about 100;

A² is selected from the group consisting of

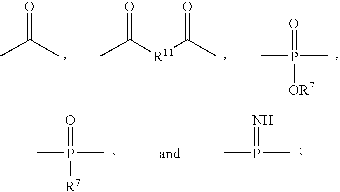

wherein R⁷ is H or a C₁ to C₃₀ hydrocarbon and R¹¹ is selected from the group consisting of C₁-C₃₀ alkyl, C₁-C₃₀ heteroalkyl, C₅-C₃₀ aryl, C₆-C₃₀ alkylaryl, and C₂-C₃₀ heteroaryl. In an embodiment, B is an aliphatic linear or branched diol or a poly(alkylene glycol) unit.

Another embodiment provides an inherently radiopaque, biocompatible, bioabsorbable polymer, wherein the polymer comprises one or more recurring units of the Formula (XII) and one or more recurring units of the Formula (XIII), each as defined above, and further comprises one or more recurring units of the Formula (XIV):

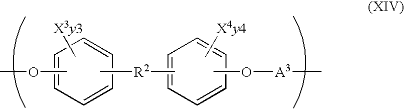

wherein:

X³ and X⁴ are each independently selected from the group consisting of Br and I;

y3 and y4 are each independently zero or an integer in the range of 1 to 4;

R² is selected from the group consisting of

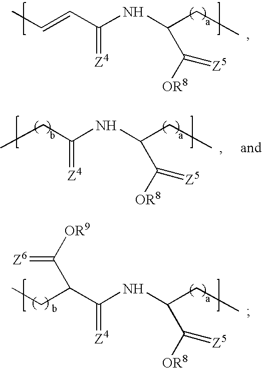

R⁸ and R⁹ are each independently H or a non-crystallizable C₁ to C₃₀ hydrocarbon;

Z⁴, Z⁵ and Z⁶ are each independently O or S;

a and b are each independently an integer in the range of 1 to 8;

A³ is selected from the group consisting of

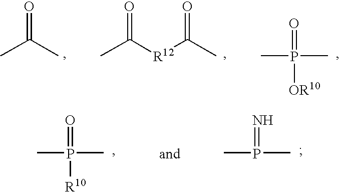

wherein R¹⁰ is selected from the group consisting of H, C₁-C₃₀ alkyl, and C₁-C₃₀ heteroalkyl; and wherein R¹² is selected from the group consisting of C₁-C₃₀ alkyl, C₁-C₃₀ heteroalkyl, C₅-C₃₀ aryl, C₆-C₃₀ alkylaryl, and C₂-C₃₀ heteroaryl. Another embodiment provides a medical device that comprises such a polymer.

In certain embodiments, the polymer may comprise one or more recurring units of the formulae (XII), (XIII), and/or (XIV). For example, another embodiment provides an inherently radiopaque, biocompatible, bioresorbable polymer, wherein the polymer comprises on or more recurring units of the Formula (XV):

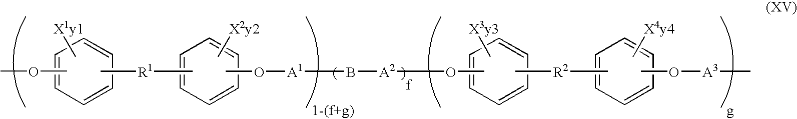

wherein X¹, X², X³, X⁴, y1, y2, y3, y4, R¹, R², A¹, A², A³ and B are as defined above, and wherein f and g may each independently range from 0 to 1, e.g., as compositional/performance requirements dictate, with the provisio that the sum of f and g is less than 1.

Any of the embodiments can advantageously be coated with a swelling material (e.g., hydrogels) and/or therapeutic agents which can promote tissue growth and/or thrombosis to assist the base device to occlude the aneurysm or other cavity. In some embodiments, the filament members have a differential cross-section (for example, notched) at various points along their length. In other embodiments, the filament members have a substantially constant cross section. The differential and constant cross section embodiments allow for selection to suit a particular need such as in connection with pushability, flexibility and detachment method of the device.

In one embodiment, an embolic filament is disclosed for occluding an aneurysm. The filament preferably comprises a bioresorbable radiopaque material as described above. The material may comprise a radiopaque polymer. In a variation, the material may comprise an erodible or corrodible metal. In one preferred embodiment, the filament further comprises notches configured to facilitate detachment of the filament.

A device for deploying an embolic filament to an aneurysm is disclosed in accordance with another preferred embodiment. The device may comprise a guiding catheter with a lumen adapted for endoluminal catheterization of the aneurysm; a spooling mechanism comprising a length of the embolic filament wound around a spool; a filament advancing mechanism adapted to advance the filament distally through the guiding catheter; and filament detachment mechanism adapted to sever the advancing filament thereby facilitating filament deployment within the aneurysm.

In a preferred variation, the device may further comprise a compliant balloon configured to bridge the aneurysm neck.

A method for embolizing a vascular aneurysm is also disclosed. The method comprises providing the above-described device; catheterizing the aneurysm; engaging the filament advancing mechanism; and engaging the filament detachment mechanism.

An embolic filament bundle for occluding an aneurysm is disclosed in accordance with another embodiment of the present invention. The embolic filament bundle comprises a plurality of embolic filaments and a bundled section where the filaments are bundled together at a predetermined location. Preferably, the bundled section is shaped to facilitate deployment without causing perforation of the aneurysm.

A device for deploying the embolic filament bundle is also disclosed. The device comprises a guiding catheter with a lumen adapted for endoluminal catheterization of the aneurysm; and a pusher rod for advancing the embolic filament bundle distally through the guiding catheter thereby facilitating embolic filament bundle deployment within the aneurysm.

A method for embolizing a vascular aneurysm using embolic filament bundles is also disclosed. The method comprises providing the above-described bundle deployment device; catheterizing the aneurysm; loading at least one embolic filament bundle into the device; and advancing the pusher rod thereby deploying the embolic filament bundle.

In addition to treating aneurysms, other examples of the use of an implantable embolic medical device comprising a non-erodible, erodible or biodegradable material, include but are not limited to the control of bleeding, prevention of blood loss prior to or during a surgical procedures, restriction or blocking of blood supply to tumors (i.e., chemo-embolization), and vascular malformations (e.g., uterine fibroids), hemorrhage (e.g., during trauma with bleeding), and arteriovenous malformations and fistulas (e.g., AVF's).

For purposes of summarizing the invention, certain aspects, advantages and novel features of the invention have been described herein above. Of course, it is to be understood that not necessarily all such advantages may be achieved in accordance with any particular embodiment of the invention. Thus, the invention may be embodied or carried out in a manner that achieves or optimizes one advantage or group of advantages as taught or suggested herein without necessarily achieving other advantages as may be taught or suggested herein.

All of these embodiments are intended to be within the scope of the invention herein disclosed. These and other embodiments of the invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

Having thus summarized the general nature of the invention and some of its features and advantages, certain preferred embodiments and modifications thereof will become apparent to those skilled in the art from the detailed description herein having reference to the figures that follow, of which:

FIG. 1 is a simplified schematic view of a lateral wall aneurysm formed by outward bulging of a blood vessel wall.

FIG. 2 is a simplified schematic view of a bifurcated aneurysm formed at the junction of a plurality of blood vessels with an embolic prosthesis in an early stage of deployment having features and advantages in accordance with an embodiment of the invention.

FIG. 3 is a simplified lengthwise-sectional view of a non-notched embolic filament having features and advantages in accordance with an embodiment of the invention.

FIG. 4 is a simplified lengthwise-sectional view of a notched embolic filament having features and advantages in accordance with another embodiment of the invention.

FIG. 5 is a simplified lengthwise-sectional view of a double-notched embolic filament having features and advantages in accordance with yet another embodiment of the invention.

FIG. 6 is a simplified schematic view of an embolic filament spool device advancing an embolic filament to an aneurysm site having features and advantages in accordance with an embodiment of the invention.

FIG. 7A is a simplified schematic enlarged view of a filament advancement mechanism of the spool device of FIG. 6 having features and advantages in accordance with an embodiment of the invention. FIG. 7B shows a motorized spool device.

FIG. 8 is a simplified schematic view of a dual lumen pressurized guiding catheter for fracturing an embolic filament proximate a distal tip of the catheter having features and advantages in accordance with an embodiment of the invention.

FIG. 9 is a simplified sectional view along line 10-10 of FIG. 8 illustrating a dual lumen configuration having features and advantages in accordance with an embodiment of the invention.

FIG. 10 is a simplified sectional view along line 11-11 of FIG. 8 illustrating a dual lumen configuration having features and advantages in accordance with another embodiment of the invention.

FIG. 11 a simplified enlarged lengthwise-sectional view of the guiding catheter and embolic filament of FIG. 8 illustrating the controlled tolerance placement of the filament within the catheter internal lumen having features and advantages in accordance with an embodiment of the invention.

FIG. 12 is a simplified schematic view of the dual lumen pressurized guiding catheter of FIG. 8 illustrating detachment of the embolic filament having features and advantages in accordance with an embodiment of the invention.

FIG. 13 is a simplified schematic enlarged of region A-A of FIG. 12 illustrating pressurized detachment of the embolic filament in progress having features and advantages in accordance with an embodiment of the invention.

FIG. 14 is a simplified schematic view of a dual lumen cutting and guiding catheter for fracturing an embolic filament proximate a distal tip of the catheter having features and advantages in accordance with another embodiment of the invention.

FIG. 15 is a simplified schematic view of a plurality of bundled embolic prostheses deployed in an aneurysm having features and advantages in accordance with an embodiment of the invention.

FIG. 16 is a simplified schematic side view of a bundled embolic prosthesis with variable length mono filaments having features and advantages in accordance with an embodiment of the invention.

FIG. 17 is a simplified schematic view of the bundled embolic prosthesis of FIG. 18 with an end bonding configuration having features and advantages in accordance with an embodiment of the invention.

FIG. 18 is a simplified schematic view of the bundled embolic prosthesis of FIG. 16 with a middle section bonding configuration having features and advantages in accordance with another embodiment of the invention.

FIG. 19 is a simplified schematic view of the bundled embolic prosthesis of FIG. 16 in a non-coiled extended state illustrating its overall length.

FIG. 20 is a simplified schematic view of a distal end of a mono filament of the bundled embolic prosthesis of FIG. 16 having features and advantages in accordance with an embodiment of the invention.

FIG. 21 is a simplified schematic view of two bundled embolic prostheses that are serially connected having features and advantages in accordance with an embodiment of the invention.

### DETAILED DESCRIPTION OF THE PREFERRED EMBODIMENTS

The preferred embodiments of the invention described herein relate generally to medical systems and methods for forming an occlusion in a mammalian body and, in particular, to systems and methods for the treatment of vascular aneurysms, preferably neurovascular aneurysms, with an implantable embolic device with one or more filaments that can be materials, such as polymers and metals, that are resorbable, non-resorbable, erodible, non-erodible, radiopaque, non-radiopaque, and that can comprise shape memory materials, swelling material (e.g., hydrogels) and/or therapeutic agents, and combinations thereof.

In addition to treating aneurysms other examples of the use of this implantable embolic medical device comprising a non-erodible, erodible or biodegradable material includes but are not limited to the control bleeding, prevention of blood loss prior to or during a surgical procedure, restriction or blocking of blood supply to tumors and vascular malformations, e.g., for uterine fibroids, tumors (i.e., chemo-embolization), hemorrhage (e.g., during trauma with bleeding) and arteriovenous malformations and fistulas (e.g., AVF's).

One skilled in the art will recognize that the embodiment described herein may be applied into any body lumen or cavity of a mammal in an amount that is effective to at least partially occlude the body cavity. In general, such a method may be used to occlude any type body cavity including, e.g., various body cavities that may commonly be referred to as tubes, tubules, ducts, channels, foramens, vessels, voids, and canals. In a preferred embodiment, the medical device is an embolotherapy product. In another preferred embodiment, the body cavity comprises vasculature, e.g., an arteriovenous malformation or a blood vessel such as a varicose vein.

While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described herein.

The methods which are described and illustrated herein are not limited to the sequence of acts described, nor are they necessarily limited to the practice of all of the acts set forth. Other sequences of acts, or less than all of the acts, or simultaneous occurrence of the acts, may be efficaciously utilized in practicing embodiments of the invention.

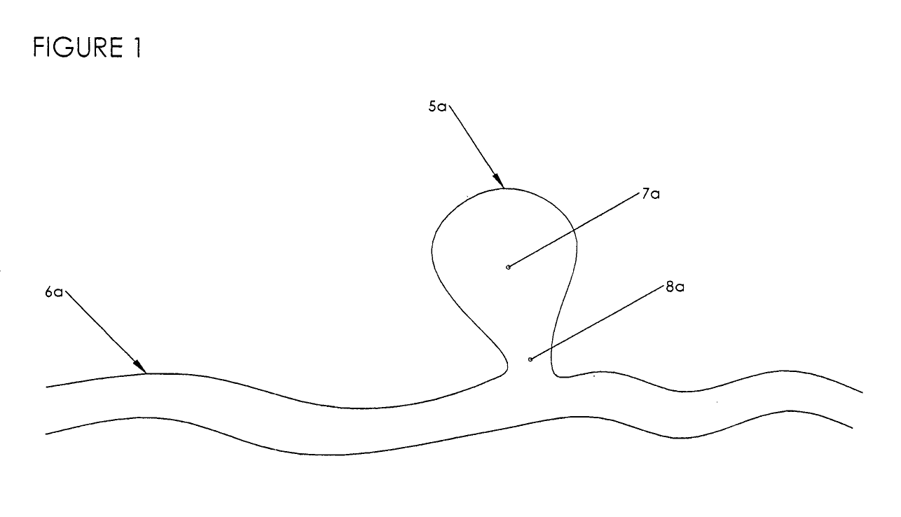

FIG. 1 schematically illustrates neurovascular morphology. FIG. 1 shows a lateral wall aneurysm 5a extending from a blood vessel 6a. The neurovascular or cerebral aneurysm 5a generally comprises a sac 7a and has a neck 8a.

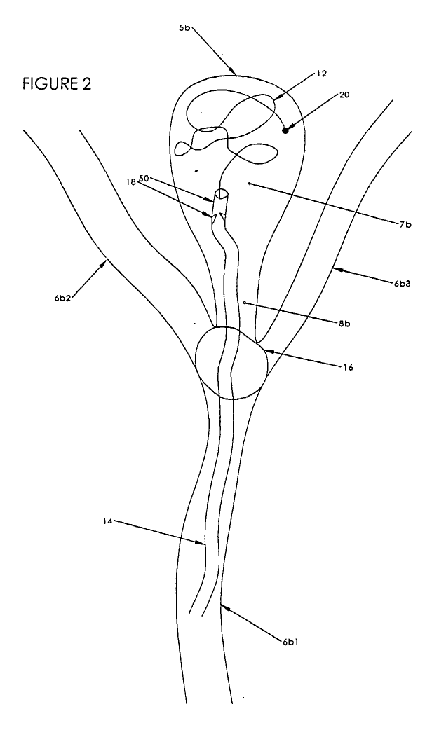

FIG. 2 shows a bifurcated aneurysm 5b extending from a junction where a blood vessel 6b1 bifurcates into vessels 6b2 and 6b3. The neurovascular or cerebral aneurysm 5b generally comprises a sac 7b and has a neck 8b.

The aneurysms 5 are formed by the bulging of blood vessels 6 to form a sack like shape. These aneurysms 5 are typically referred to as saccular aneurysms. Embodiments of the invention have particular efficacy in treating saccular aneurysms 5 though in modified embodiments other types of aneurysms may be treated with efficacy, such as, but not limited to, fusiform aneurysms which are formed by bulging of the blood vessel over substantially its entire cross section or circumference.

### Embolic Filament Embodiment

Some embodiments relate to, but are not limited to, the design, manufacture and use of embolic filaments to occlude aneurysms in the neurovasculature or other sites where embolization is required to satisfy a particular clinical objective. These longitudinal filament members are designed to have a longitudinal profile and cross sectional geometry along their length such that they are substantially matched to two parameters. One is the mechanical properties of the embolic filament material and the second is the precise clearance dimensions (clearance gap) between the embolic filament and the delivery conduit to enable filament flexibility while maintaining the filaments “pushability” to reach the target embolic site (which in the case of the treatment of neurovascular aneurysms, can be located at distal and tortuous locations deep within the neurovasculature).

This precise clearance gap (defined as the internal dimension of the delivery conduit minus the outside dimension of the embolic filament), when sized appropriately through engineering calculation and experimentation, will allow the embolic device to have dimensions in between the outside dimension of the embolic filament and the inside dimension of the delivery conduit. The embolic filaments can be made from a number of suitable materials with each particular material having a specific set of mechanical properties. Advantageously, this allows optimization and/or customization to the appropriate amount of pushability and flexibility to enable the embolic device to reach the aneurysm and to fill the aneurysm to occlude the neck without rupturing the aneurysm during the process.

FIG. 2 shows a partial view of an apparatus or system 10 including an embolic filament or device 12 being deployed in the aneurysm 5b utilizing a guiding catheter 14. As discussed further below, a preferably low durometer compliant balloon 16 is used to bridge the aneurysm neck 8b.

FIG. 2 also shows an area of detachment 18 of the filament 12 relative to the catheter 14. As discussed in more detail below, the guiding catheter 14 is used to perform the detachment once the filament 12 densely packs the aneurysm 5b. One embodiment involves the introduction of pressure to create tensile stress on a necked down embolic filament. Another embodiment involves the use of a hydraulically actuated cutting mechanism.

The embolic filament 12 comprises a suitably strong and flexible material that can be advanced through the catheter 14 and densely pack the aneurysm 5b to occlude or embolize it. The filament member 12 comprises a longitudinal member which terminates in a distal tip 20 that is substantially blunt or rounded to avoid puncture and subsequent rupture of the aneurysm 5b.

The filament 12 can be fabricated by any one of a number of manufacturing techniques. For example when using metal, the filament 12 can be made by a hot or cold drawing process. In the case of polymer filament, the filament 12 can be made by an extrusion process and secondary hot or cold drawing process.

In some embodiments, the filament 12 comprises radiopaque or non-radiopaque polymers. In some embodiments, the filament 12 comprises biodegradable, degradable or non-resorbable polymers. Preferred bioresorbable radiopaque polymers are disclosed in U.S. Pat. No. 6,475,477, and co-pending U.S. application Ser. Nos. 10/952,202, 10/952,274, 11/176,638, 11/200,656 and 11/335,771; all of which are incorporated herein in their entirety by reference thereto. Preferably, the bioresorbable radiopaque polymers are selected from the following generic structures (formulas I-XV).

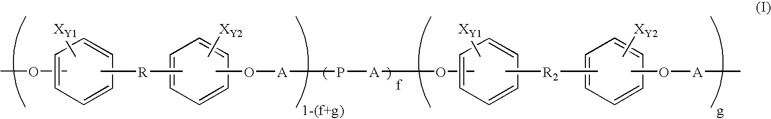

wherein each X is independently I or Br, Y1 and Y2 for each diphenol unit are independently between 0 and 4, inclusive, and Y1+Y2 for each diphenol unit is between 1 and 8, inclusive.

wherein each R and R2 are independently an alkyl, aryl or alkylaryl group containing up to 18 carbon atoms and from 0 to 8 heteroatoms selected from O and N and R2 further comprises a pendant free carboxylic acid group;

wherein A is either:

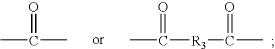

wherein R3 is a saturated or unsaturated, substituted or unsubstituted alkyl, aryl, or alkylaryl group containing up to about 18 carbon atoms and 0 to 8 heteroatoms selected from O and N;

wherein P is a poly(C1-C4 alkylene glycol) unit; f is from 0 to less than 1; g is from 0 to 1, inclusive; and f+g ranges from 0 to about 1, inclusive.

Preferably, iodine and bromine are both present as ring substituents. Further, all X groups are preferably ortho-directed. Y1 and Y2 may independently be 2 or less, and Y1+Y2=1, 2, 3 or 4. In another variation, Y1+Y2=2 or 3. All X groups are preferably iodine.

In another variation to the present invention, the weight fraction of the poly(C1-C4 alkylene glycol) unit is less than about 75 wt %. In a preferred variation, the weight fraction of the poly(C1-C4 alkylene glycol) unit is less than about 50 wt %. More preferably, the poly(C1-C4 alkylene glycol) is poly(ethylene glycol) with a weight fraction of less than about 40 wt %. Most preferably, the weight fraction of the poly(ethylene glycol) unit is between about 1 and 25 wt %. P may independently be C1 up to C4 or copolymers of C1-C4.

In another variation to the present invention, f may vary between about 0 and 0.5, inclusive. Preferably, f is less than about 0.25. More preferably, f is less than about 0.1. More preferably yet, f varies from about 0.001 to about 0.08. Most preferably, f varies between about 0.025 and about 0.035.

In another variation to the present invention, g is greater than 0 and typically varies between greater than 0 and about 0.5, inclusive. Preferably, g is greater than about 0.1 to about 0.35. More preferably, g is from about 0.2 to about 0.3. More preferably yet, g varies between about 0.01 and about 0.25. Most preferably, g is between about 0.05 and about 0.15.

In another variation to the present invention, R2 further comprises a pendant carboxylic acid group. Preferably, both R and R2 comprise a pendant COOR1 group; wherein for R, the subgroup R1 is independently an alkyl group ranging from 1 to about 18 carbon atoms containing from 0 to 5 heteroatoms selected from O and N; and wherein for R2, the subgroup R1 is a hydrogen atom. In another preferred embodiment, each R and R2 independently has the structure:

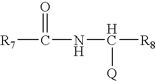

wherein R7 is selected from the group consisting of —CH═CH—, —CHJ1-CHJ2- and (—CH2-)a; wherein R8 is selected from the group consisting of —CH═CH—, —CHJ1-CHJ2- and (—CH2-)n; wherein a and n are independently between 0 and 8 inclusive; and J1 and J2 are independently Br or I; and wherein, for each R2, Q comprises a free carboxylic acid group, and for each R, Q is independently selected from the group consisting of hydrogen and carboxylic acid esters and amides, wherein said esters and amides are selected from the group consisting of esters and amides of alkyl and alkylaryl groups containing up to 18 carbon atoms and esters and amides of biologically active compounds.

In a preferred variation to the present invention, each R and R2 independently has the structure:

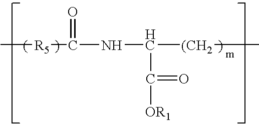

wherein R5 is an alkyl group containing up to 18 carbon atoms and from 0 to 5 heteroatoms selected from O and N; and wherein m is an integer from 1 to 8 inclusive; and wherein, for each R2, R1 is hydrogen, and, for each R, R1 is independently an alkyl group ranging from 1 to about 18 carbon atoms containing from 0 to 5 heteroatoms selected from O and N.

In a more preferred variation to the present invention, each R and R2 independently has the structure:

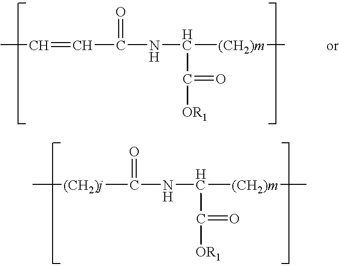

wherein j and m are independently an integer from 1 to 8, inclusive, and wherein, for each R2, R1 is hydrogen, and, for each R, R1 is independently an alkyl group ranging from 1 to about 18 carbon atoms containing from 0 to 5 heteroatoms selected from O and N.

Preferably, each R1 subgroup for R is independently an alkyl group ranging from 1 to about 18 carbon atoms and containing from 0 to 5 heteroatoms selected from O and N. More preferably, each R1 subgroup for R is independently either ethyl or butyl.

In another variation to the present invention, A is a —C(═O)— group. Alternatively, A may be:

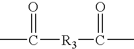

wherein R3 is a C4-C12 alkyl, C8-C14 aryl, or C8-C14 alkylaryl. Preferably, R3 is selected so that A is a moiety of a dicarboxylic acid that is a naturally occurring metabolite. More preferably, R3 is selected from the group consisting of —CH2-C(═O)—, —CH2-CH2-C(═O)—, —CH═CH— and (—CH2-)z; and wherein z is an integer from 0 to 8, inclusive. More preferably, z is an integer from 1 to 8, inclusive.

In one preferred embodiment, the filament members may comprise a polymer described in Ser. No. 10/952,274 as having one or more units described by Formula II:

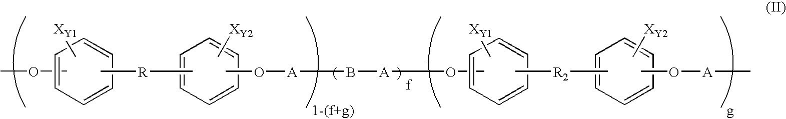

wherein X=I or Br; Y1 and Y2 can independently=0, 1, 2, 3 or 4;

wherein f is between 0 and less than 1; g is between 0 and 1, inclusive; and f+g is between 0 and 1, inclusive;

wherein A is either:

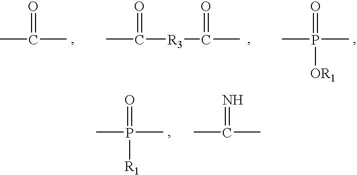

wherein R₁ is independently an H or an alkyl group ranging from 1 to about 18 carbon atoms containing from 0 to 5 heteroatoms selected from O and N;

wherein R₃ is a saturated or unsaturated, substituted or unsubstituted alkyl, aryl, or alkylaryl group containing up to about 18 carbon atoms and 0 to 8 heteroatoms selected from O and N;

wherein B is an aliphatic linear or branched diol or a poly(alkylene glycol) unit; and

wherein R and R₂ may be independently selected from:

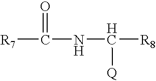

wherein R₇ is selected from the group consisting of —CH═CH—, —CHJ₁-CHJ₂- and (—CH₂-)a; wherein R₈ is selected from the group consisting of —CH═CH—, —CHJ₁-CHJ₂- and (—CH₂-)n; wherein a and n are independently between 0 and 8 inclusive; J₁ and J₂ are independently Br or I; and, for R₂, Q comprises a free carboxylic acid group, and, for R, Q is selected from the group consisting of hydrogen and carboxylic acid esters and amides, wherein said esters and amides are selected from the group consisting of esters and amides of alkyl and alkylaryl groups containing up to 18 carbon atoms and esters and amides of biologically and pharmaceutically active compounds.

In a variation to this embodiment of Formula II, R and R₂ may be selected from the groups:

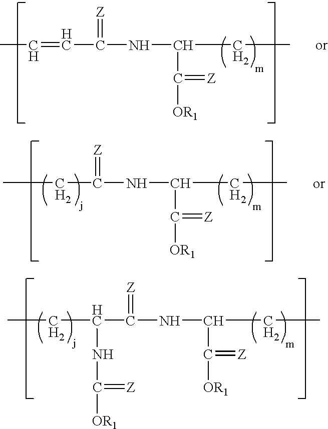

wherein R₁ in each R₂ is independently an alkyl group ranging from 1 to about 18 carbon atoms containing from 0 to 5 heteroatoms selected from O and N and R₁ in each R is H;

wherein j and m are independently integers from 1 to 8 inclusive; and

wherein Z is independently either O or S.

In another preferred embodiment, the polymer may comprise one or more units described by Formula III:

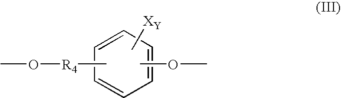

wherein X for each polymer unit is independently Br or I, Y is between 1 and 4, inclusive and R₄ is an alkyl, aryl or alkylaryl group with up to 18 carbon atoms and from 0 to 8 heteroatoms selected from O and N.

In variations to the polymer of Formula III, all X groups may be orthodirected and Y may be 1 or 2. In another variation, R₄ is an alkyl group.

In another variation, R₄ has the structure:

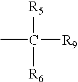

wherein R₉ for each unit is independently an alkyl, aryl or alkylaryl group containing up to 18 carbon atoms and from 0 to 8 heteroatoms selected from O and N; and R₅ and R₆ are each independently selected from hydrogen and alkyl groups having up to 18 carbon atoms and from 0 to 8 heteroatoms selected from O and N.

In another variation to R₄ in Formula III, R₉ for at least one unit comprises a pendant COOR₁ group, wherein, for each unit in which it is present, the subgroup R₁ is independently a hydrogen or an alkyl group ranging from 1 to about 18 carbon atoms containing from 0 to 5 heteroatoms selected from O and N.

In another variation to R₄ in Formula III, R₉ independently has the structure:

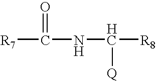

wherein R₇ is selected from the group consisting of —CH═CH—, —CHJ₁-CHJ₂- and (—CH₂-)a, wherein R₈ is selected from the group consisting of —CH═CH—, —CHJ₁-CHJ₂- and (—CH₂-)n, wherein a and n are independently between 0 and 8 inclusive; and J₁ and J₂ pendently Br or I; and Q is selected from the group consisting of hydrogen, a free carboxylic acid group, and carboxylic acid esters and amides, wherein said esters and amides are selected from the group consisting of esters and amides of alkyl and alkylaryl groups containing up to 18 carbon atoms and esters and amides of biologically and pharmaceutically active compounds.

In another variation to R₄ in Formula III, R₉ independently has the structure:

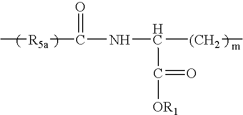

wherein R₅ₐ is an alkyl group containing up to 18 carbon atoms and from 0 to 5 atoms selected from O and N; and wherein m is an integer from 1 to 8 inclusive; and R₁ is independently a hydrogen or an alkyl group ranging from 1 to about 18 carbon atoms containing from 0 to 5 heteroatoms selected from O and N.

In another variation to R₄ in Formula III, R₉ independently has the structure:

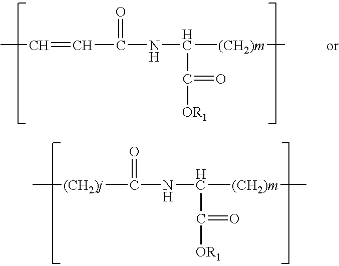

wherein j and m are independently an integer from 1 to 8, inclusive, and R₁ is independently a hydrogen or an alkyl group ranging from 1 to about 18 carbon atoms containing from 0 to 5 heteroatoms selected from O and N.

In some embodiments, the polymer may be copolymerized with a poly(C₁-C₄ alkylene glycol). Preferably, the poly(C₁-C₄ alkylene glycol) is present in a weight fraction of less than about 75 wt %. More preferably, the poly(alkylene glycol) is poly(ethylene glycol).

In another variation to the polymers disclosed herein, between about 0.01 and about 0.99 percent of said polymer units comprise a pendant —COOH group.

In another variation to Formula III, R₄ may be an aryl or alkylaryl group. Preferably, the R₄ aryl or alkylaryl group is selected so that the polymer units are diphenols.

In another preferred embodiment, the polymer may comprise one or more units described by Formula IV:

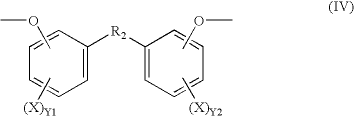

wherein X for each polymer unit is independently Br or I, Y1 and Y2 are each independently between 0 and 4, inclusive, Y1+Y2 for each unit is independently between 1 and 8, inclusive, and R₂ for each polymer unit is independently an alkyl, aryl or alkylaryl group containing up to 18 carbon atoms and from 0 to 8 heteroatoms selected from O and N.

In preferred variations to Formula IV, all X groups are ortho-directed. Preferably, Y1 and Y2 are independently 2 or less, and Y1+Y2=1, 2, 3 or 4.

In another variation to Formula IV, R₂ for at least one unit may comprise a pendant COOR₁ group, wherein, for each unit in which the COOR₁ group is present, the subgroup R₁ is independently a hydrogen or an alkyl group ranging from 1 to about 18 carbon atoms containing from 0 to 5 heteroatoms selected from O and N.

In another variation to Formula IV, R₂ independently has the structure:

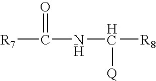

wherein R₇ is selected from the group consisting of —CH═CH—, —CHJ₁-CHJ₂- and (—CH₂-)a, wherein R₈ is selected from the group consisting of —CH═CH—, —CHJ₁-CHJ₂- and (—CH₂-)n, wherein a and n are independently between 0 and 8 inclusive; and J₁ and J₂ pendently Br or I; and Q is selected from the group consisting of hydrogen, a free carboxylic acid group, and carboxylic acid esters and amides, wherein said esters and amides are selected from the group consisting of esters and amides of alkyl and alkylaryl groups containing up to 18 carbon atoms and esters and amides of biologically and pharmaceutically active compounds.

In another variation to Formula IV, R₂ independently has the structure:

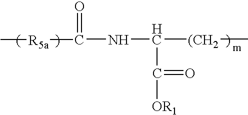

wherein R₅ₐ is an alkyl group containing up to 18 carbon atoms and from 0 to 5 heteroatoms selected from O and N; and wherein m is an integer from 1 to 8 inclusive; and R₁ is independently a hydrogen or an alkyl group ranging from 1 to about 18 carbon atoms containing from 0 to 5 heteroatoms selected from O and N.

In another variation to Formula IV, R₂ independently has the structure:

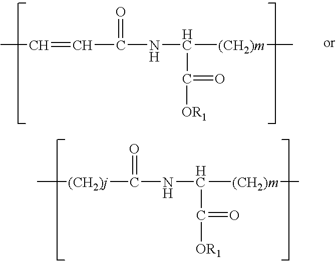

wherein j and m are independently an integer from 1 to 8, inclusive, and R₁ is independently a hydrogen or an alkyl group ranging from 1 to about 18 carbon atoms containing from 0 to 5 heteroatoms selected from O and N.

In a preferred variation to Formula IV, between about 0.01 and about 0.99 percent of the polymer units comprise a pendant COOH group. Preferably, the polymer is copolmerized with up to 75 wt % of a poly(C₁-C₄ alkylene glycol). More preferably, the poly(C₁-C₄ alkylene glycol) is poly(ethylene glycol).

In another preferred embodiment, the polymer may comprise one or more by Formula V:

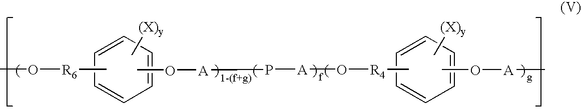

wherein each X is independently iodine or bromine; each y is independently between 0 and 4, inclusive, wherein a total number of ring-substituted iodine and bromine is between 1 and 8, inclusive;,each R₄ and R₆ are independently an alkyl, aryl or alkylaryl group containing up to 18 carbon atoms and from 0 to 8 heteroatoms selected from O and N, and R₄ further includes a pendant carboxylic acid group;

wherein A is either:

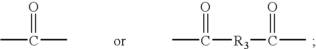

wherein R₃ is a saturated or unsaturated, substituted or unsubstituted alkyl, aryl, or alkylaryl group containing up to about 18 carbon atoms and 0 to 5 heteroatoms selected from the group consisting of O and N;

P is a poly(C₁-C₄ alkylene glycol) unit present in a weight fraction of less than about 75 wt %;

f is from greater than 0 to less than 1; g is between 0 and 1, inclusive; and f+g is between 0 and 1, inclusive.

Preferably, P is a poly(ethylene glycol) unit.

In preferred variations to Formula V, each R₄ and R₆ of said polymer contains a pendant —COOR₁ group, wherein for each R₆, each subgroup R₁ is independently an alkyl group ranging from 1 to about 18 carbon atoms containing from 0 to 5 heteroatoms selected from the group consisting of O and N, and, for each R₄, each subgroup R₁ is a hydrogen atom.

In other preferred variations to Formula V, each R₄ and R₆ of said polymer are:

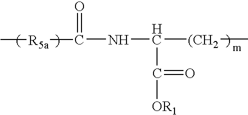

wherein R₅ₐ is an alkyl group containing up to 18 carbon atoms and from 0 to 5 heteroatoms selected from O and N; and wherein m is an integer from 1 to 8 inclusive; and for each R₆, each subgroup R₁ is independently an alkyl group ranging from 1 to about 18 carbon atoms containing from 0 to 5 heteroatoms selected from O and N, and, for each R₄, each subgroup R₁ is a hydrogen atom.

In other preferred variations to Formula V, each R₁ subgroup for R₆ of said polymer is either ethyl or butyl.

In other preferred variations to Formula V, A is a —C(═O)— group. Alternatively, A may be:

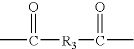

wherein R₃ is C₄-C₁₂ alkyl, C₈-C₁₄ aryl, or C₈-C₁₄ alkylaryl.

In other preferred variations to Formula V, R₃ is selected so that A is a moiety of a dicarboxylic acid that is a naturally occurring metabolite.

In other preferred variations to Formula V, R₃ is a moiety selected from the group consisting of —CH₂—C(═O)—, —CH₂—CH₂—C(═O)—, —CH═CH— and (—CH₂-)z, wherein z is an integer from 1 to 8, inclusive.

In other preferred variations to Formula V, all X groups are ortho-directed and y is 2 or 3.

In other preferred variations to Formula V, every X group is iodine.

In other preferred variations to Formula V, f is greater than 0.1 to about 0.3.

In other preferred variations to Formula V, g is greater than 0.1 to about 0.35.

In one preferred embodiment, the filament members may comprise an inherently radiopaque side chain crystallizable polymer, comprising a main chain, a plurality of crystallizable side chains, and a plurality of heavy atoms attached to the polymer, the heavy atoms being present in an amount that is effective to render the polymer radiopaque. A polymer that comprises a recurring unit of the formula (VI) is an example of such an inherently radiopaque side chain crystallizable polymer:

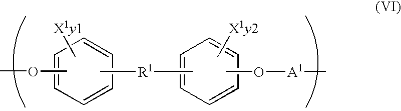

In formula (VI), X¹ and X² are each independently selected from the group consisting of Br and I; y¹ and y² are each independently zero or an integer in the range of 1 to 4; and A¹ is selected from the group consisting of

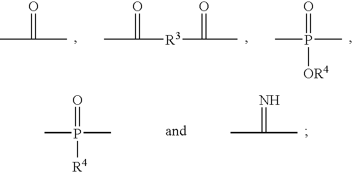

R³ is selected from the group consisting of C₁-C₃₀ alkyl, C₁-C₃₀ heteroalkyl, C₅-C₃₀ aryl, C₆-C₃₀ alkylaryl, and C₂-C₃₀ heteroaryl; R⁴ selected from the group consisting of H, C₁-C₃₀ alkyl, and C₁-C₃₀ heteroalkyl; R¹ is

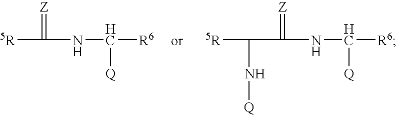

R⁵ and R⁶ are each independently selected from the group consisting of —CH═CH—, —CHJ¹, —CHJ²—, and —(CH₂)ₐ—; a is zero or an integer in the range of 1 to 8; J¹ and J² are each independently selected from the group consisting of Br and I; and Z is an O or an S; and Q is a cryatallizable group comprising from about 6 to about 30 carbon atoms, preferably from about 20 to about 30 carbon atoms. In an embodiment, Q is:

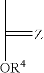

Polymers of the formula (VI) may be prepared by modifying the general methods described in U.S. patent application Ser. No. 11/200,656, to select the appropriate side chain length, side chain spacing and halogen content.

It will be recognized that Q and/or R⁴ may comprise crystallizable side chains, that each of X, J¹ and J² is a heavy atom, and that y may be adjusted so that the number of heavy atoms in the polymer is sufficient to render the polymer radiopaque. Q and R⁴ may each independently comprise units selected from the group consisting of —(CH₂)ₙ₁— and —((CH₂)ₘ₁—O—)ₙ₁; where m1 and n1 are each independently selected so that Q and/or R⁴ each independently contain from about 1 to about 30 carbon atoms, preferably from about 6 to about 30 carbon atoms, and more preferably from about 20 to 30 carbon atoms. Moreover, Q and R⁴ may include other functional groups such as ester and amide, and/or heavy atoms such as iodine and bromine. Non-limiting examples of Q and R⁴ thus include —Cₙ₁H₂ₙ₁₊₁, —CO₂—Cₙ₁H₂ₙ₁₊₁, —CONH—Cₙ₁H₂ₙ₁₊₁, —(CH₂)ₙ₁—Br, —(CH₂)ₙ₁—I, —CO₂—(CH₂)ₙ₁—Br, —CO₂—(CH₂)ₙ₁—I, —CONH—CO₂—(CH₂)ₙ₁—Br, and —CONH—CO₂—(CH₂)ₙ₁—I. In an embodiment, R⁵ is —CH═CH— or —(CH₂)ₐ—; R⁶ is —(CH₂)ₐ—; and Q is an ester group comprising from about 10 to about 30 carbon atoms.

It will be understood that a polymer that comprises a recurring unit of the formula (I) may be a copolymer, e.g., a polymer of the formula (I) that further comprises recurring —R²-A²- units, where R² is selected from the group consisting of —(CH₂)ₙ₂— and —((CH₂)ₘ₂—O—)ₙ₂; where m2 and n2 are each independently selected so that R² contains from about 1 to about 30 carbon atoms; and where A² is defined in the same manner as A¹ above. Thus, an embodiment provides a polymer comprising recurring units of the formula (VIa):

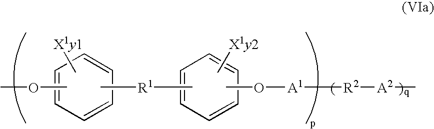

In formula (VIa), X¹, X², y¹, y², R¹ and A¹ are defined as described above for formula (VI); p and q may each be independently varied over a broad range to provide a polymer having the desired properties, e.g., melting point, radiopacity, and viscosity, using routine experimentation. In an embodiment, p and q are each independently an integer in the range of 1 to about 10,000. It will be appreciated that the formula (VI) units and —R²-A²)- units in a polymer comprising recurring units of the formula (VIa) may be arranged in various ways, e.g., in the form of a block copolymer, random copolymer, alternating copolymer, etc.

Another embodiment of an inherently radiopaque side chain crystallizable polymer (e.g., a polymer comprising a main chain, a plurality of crystallizable side chains, and a plurality of heavy atoms attached to the polymer, the heavy atoms being present in an amount that is effective to render the polymer radiopaque), comprises a recurring unit of the formula (VII):

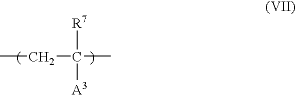

In formula (VII), R⁷ is H or CH₃; A³ is a chemical group having a molecular weight of about 500 or less; and A³ bears at least one of the heavy atoms attached to the polymer. Non-limiting examples of A³ include metal carboxylate (e.g., —CO₂Cs), metal sulfonate (e.g., —SO₄Ba), halogenated alkyl ester (e.g., —CO₂—(CH₂)_{b}—Br), halogenated alkyl amide (e.g., —CONH—(CH₂)_{b}—Br), and halogenated aromatic (e.g., —C₆H₄—I), where b is an integer in the range of about 1 to about 4. In an embodiment, A³ comprises an aromatic group bearing at least one halogen atom selected from the group consisting of bromine and iodine. In another embodiment, A³ comprises a chemical group of the formula -L₁-(CH₂)ₙ₃-L₂-Ar¹, wherein L₁ and L₂ each independently represent a nullity (i.e., are not present), ester, ether or amide group; n3 is zero or an integer in the range of about 1 to about 30; and Ar¹ comprises a halogenated aromatic group containing from about 2 to about 20 carbon atoms. Inherently radiopaque side chain crystallizable polymers that comprise a recurring unit of the formula (VII) may be formed by polymerization of the corresponding monomers or by post-reaction of appropriate polymeric precursors. Inherently radiopaque side chain crystallizable polymers that comprise a recurring unit of the formula (VII) may be copolymers that include additional recurring units.

Side chain A³ groups in an inherently radiopaque side chain crystallizable polymer comprising a recurring unit of the formula (VII) may be crystallizable and/or the inherently radiopaque side chain crystallizable polymer comprising a recurring unit of the formula (VII) may further comprise a second recurring unit that comprises a crystallizable side chain. Examples of suitable second recurring units having crystallizable side chains include the following: poly(1-alkene)s, poly(alkyl acrylate)s, poly(alkyl methacrylate)s, poly(alkyl vinyl ether)s, and poly(alkyl styrene)s. The alkyl groups of the foregoing exemplary second recurring units preferably contain more than 6 carbon atoms, and more preferably contain from about 6 to about 30 carbon atoms. For example, in an embodiment, the second recurring unit is of the formula (VIII):

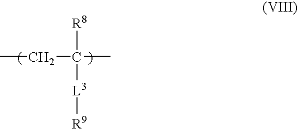

In formula (VIII), R⁸ is H or CH₃; L³ is an ester or amide linkage; and R⁹ comprises a C₆ to C₃₀ hydrocarbon group. Inherently radiopaque side chain crystallizable polymers comprising a recurring unit of the formula (VII) and a second recurring unit (such as a recurring unit of the formula (VIII)) may be formed by copolymerization of the corresponding monomers and/or by post reaction of appropriate polymeric precursors.

Another embodiment of an inherently radiopaque side chain crystallizable polymer (e.g., a polymer comprising a main chain, a plurality of crystallizable side chains, and a plurality of heavy atoms attached to the polymer, the heavy atoms being present in an amount that is effective to render the polymer radiopaque) comprises a recurring unit of the formula (IX), where A³ is defined above:

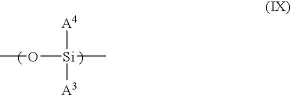

In formula (IX), A⁴ represents H or a group containing from about 1 to about 30 carbons, e.g., a C₁-C₃₀ hydrocarbon. Side chain A³ and/or A⁴ groups in an inherently radiopaque side chain crystallizable polymer may comprise a recurring unit of the formula (IX) and may further comprise a second recurring unit that comprises a crystallizable side chain. For example, in an embodiment, the second recurring unit is of the formula (X), where R¹⁰ comprises a C₆ to C₃₀ hydrocarbon group and R¹¹ represents H or a group containing from about 1 to about 30 carbons, e.g., a C₁-C₃₀ hydrocarbon:

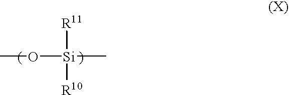

In one preferred embodiment, the filament members may comprise a polymer described in Ser. No. 11/335,771, comprising a recurring unit of the formula (XI):

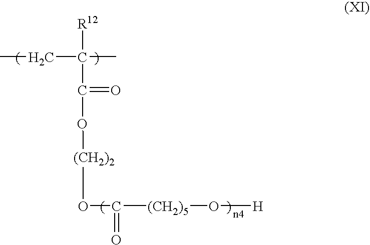

wherein R¹² is H or CH₃ and n4 is an integer in the range of about 1 to about 1,000. In preferred embodiments, the polymer comprising a recurring unit of the formula (XI) is biocompatible.

In one preferred embodiment, the filament members may comprise a polymer described in Ser. No. 11/200,656 as an inherently radiopaque, biocompatible, bioresorbable polymer, wherein the polymer comprises one or more recurring units of the Formula (XII):

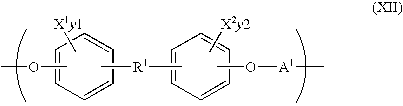

wherein:

X¹ and X² are each independently selected from the group consisting of Br and I;

y1 and y2 are each independently zero or an integer in the range of 1 to 4, with the proviso that the sum of y1 and y2 is at least one;

R¹ is

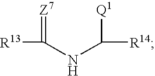

R¹³ and R¹⁴ are each independently selected from the group consisting of —CH═CH—, —(CH₂)_{c}—, —(CHJ¹)-, —CHJ²-CHJ³-, —CH═CH—(CHJ¹)-, and —CH₂)_{c}—(CHJ¹)-;

c is zero or an integer in the range of 1 to 8;

J¹, J² and J³ are each independently selected from the group consisting of H, Br, I, —NH-Q² and —C(=Z⁸)-OQ³;

Q¹, Q² and Q³ are each independently H or a non-crystallizable group comprising from about 1 to about 30 carbons;

Z⁷ and Z⁸ are each independently O or S;

A¹ is selected from the group consisting of

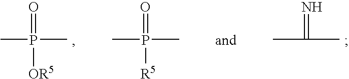

R⁵ is selected from the group consisting of H, C₁-C₃₀ alkyl, and C₁-C₃₀ heteroalkyl. In a preferred embodiment, X¹, X², y1 and y2 are selected so that X¹ and X² are present in an amount that is effective to render the polymer radiopaque.

In an embodiment of a polymer comprising a recurring unit of the Formula (XII), R¹ in Formula (XII) is:

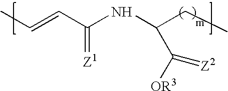

wherein R³ is H or a non-crystallizable C₁ to C₂₉ hydrocarbon;

Z¹ and Z² are each independently O or S; and

m is an integer in the range of 1 to 8.

In another embodiment of a polymer comprising a recurring unit of the Formula (XII), R¹ in Formula (XII) is:

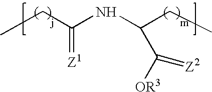

wherein R³ is H or a non-crystallizable C₁ to C₂₉ hydrocarbon;

Z¹ and Z² are each independently O or S; and

j and m are each independently an integer in the range of 1 to 8.

In another embodiment of a polymer comprising a recurring unit of the Formula (XII), R¹ in Formula (XII) is:

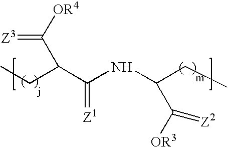

wherein R³ and R⁴ are each independently H or a non-crystallizable C₁ to C₂₉ hydrocarbon;

Z¹, Z² and Z³ are each independently O or S; and

j and m are each independently an integer in the range of 1 to 8.

Another embodiment provides a filament that comprises an inherently radiopaque, biocompatible, bioresorbable polymer, wherein the polymer comprises one or more recurring units of the Formula (XII) as described above.

Another embodiment provides an inherently radiopaque, biocompatible, bioresorbable polymer, wherein the polymer comprises one or more recurring units of the Formula (XII) as defined above, and further comprises one or more recurring units of the Formula (XIII):

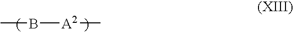

wherein:

B is —O—(CHR⁶)ₚ—O)_{q}—;

R⁶ is H or C₁ to C₃ alkyl;

p and q are each individually an integer in the range of about 1 to about 100;

A² is selected from the group consisting of

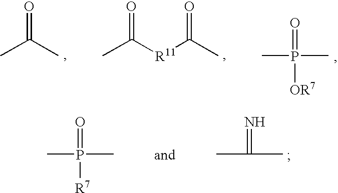

wherein R⁷ is H or a C₁ to C₃₀ hydrocarbon and R¹¹ is selected from the group consisting of C₁-C₃₀ alkyl, C₁-C₃₀ heteroalkyl, C₅-C₃₀ aryl, C₆-C₃₀ alkylaryl, and C₂-C₃₀ heteroaryl. In an embodiment, B is an aliphatic linear or branched diol or a poly(alkylene glycol) unit.

Another embodiment provides an inherently radiopaque, biocompatible, bioresorbable polymer, wherein the polymer comprises one or more recurring units of the Formula (XII) and one or more recurring units of the Formula (XIII), each as defined above, and further comprises one or more recurring units of the Formula (XIV):

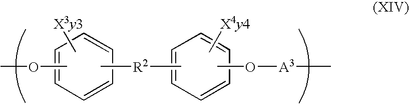

wherein:

X³ and X⁴ are each independently selected from the group consisting of Br and I;

y3 and y4 are each independently zero or an integer in the range of 1 to 4;

R² is selected from the group consisting of

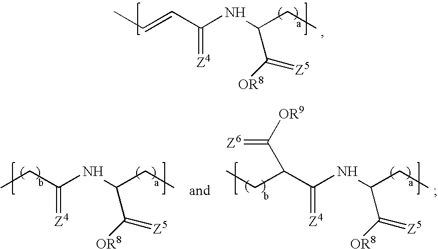

R⁸ and R⁹ are each independently H or a non-crystallizable C₁ to C₃₀ hydrocarbon;

Z⁴, Z⁵ and Z⁶ are each independently O or S;

a and b are each independently an integer in the range of 1 to 8;

A³ is selected from the group consisting of

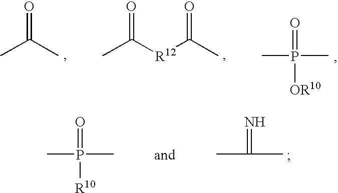

wherein R¹⁰ is selected from the group consisting of H, C₁-C₃₀ alkyl, and C₁-C₃₀ heteroalkyl; and wherein R¹² is selected from the group consisting of C₁-C₃₀ alkyl, C₁-C₃₀ heteroalkyl, C₅-C₃₀ aryl, C₆-C₃₀ alkylaryl, and C₂-C₃₀ heteroaryl. Another embodiment provides a medical device that comprises such a polymer.

In certain embodiments, the polymer may comprise one or more recurring units of the formulae (XII), (XIII), and/or (XIV). For example, another embodiment provides an inherently radiopaque, biocompatible, bioresorbable polymer, wherein the polymer comprises one or more recurring units of the Formula (XV):

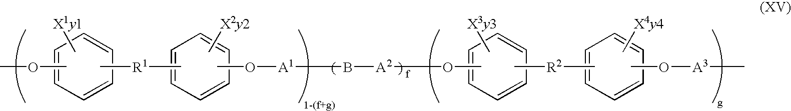

wherein X¹, X², X³, X⁴, y1, y2, y3, y4, R¹, R², A¹, A², A³ and B are as defined above, and wherein f and g may each independently range from 0 to 1, e.g., as compositional/performance requirements dictate, with the provisio that the sum of f and g is less than 1.

To the extent that those skilled in the art require particular guidance in making the above-disclosed radiopaque bioresorbable polymers, such guidance maybe found in U.S. Pat. No. 6,475,477, and co-pending U.S. application Ser. Nos. 10/952,202, 10/952,274, 11/176,638, 11/200,656 and 11/335,771; all of which are incorporated herein in their entirety by reference thereto.

In some embodiments, the filament 12 comprises erodible and corrodible or non-erodible and non-corrodible metals. In some embodiments, the filament 12 comprises shape memory metals such as, but not limited to, Nitinol and spring steel. Any combination of these embodiments may be efficaciously utilized, as needed or desired.

Biodegradable polymers are commonly known as biologic polymers with enzymatically unstable linkages in the backbone whereas and degradable polymers are generally often synthetic with hydrolytically unstable linkages in the backbone; the biodegradable and degradable polymers both resorb, i.e., resorbable materials. Non-resorbable polymers are biostable. Biodegradable and degradable polymers allow a physician to place the device that will not require a second surgical intervention for removal. These polymer devices can be engineered to degrade at a rate that will slowly transfer the mechanical load to the healing tissue. Resorbable materials (as well as corrodible or erodible metals) also offer the advantage of allowing for tissue formation in the treated space which can stabilize the aneurysm or treated cavity.

Examples of suitable degradable polymers include, but are not limited to, polyhydroxybutyrate/polyhydroxyvalerate copolymers (PHV/PHB), polyesteramides, polylactic acid, hydroxy acids (i.e. lactide, glycolide, hydroxybutyrate), polyglycolic acid, lactone based polymers, polycaprolactone, poly(propylene fumarate-co-ethylene glycol) copolymer (aka fumarate anhydrides), polyamides, polyanhydride esters, polyanhydrides, polylactic acid/polyglycolic acid with a calcium phosphate glass, polyorthesters, silk-elastin polymers, polyphosphazenes, copolymers of polylactic acid and polyglycolic acid and polycaprolactone, aliphatic polyurethanes, polyhydroxy acids, polyether esters, polyesters, polydepsidpetides, polysaccharides, polyhydroxyalkanoates, polyarylates and copolymers thereof.

In one mode, the degradable materials are selected from the group consisting of poly(glycolide-trimethylene carbonate), poly(alkylene oxalates), polyaspartimic acid, polyglutarunic acid polymer, poly-p-dioxanone, poly-.beta.-dioxanone, asymmetrically 3,6-substituted poly-1,4-dioxane-2,5-diones, polyalkyl-2-cyanoacrylates, polydepsipeptides (glycine-DL-lactide copolymer), polydihydropyranes, polyalkyl-2-cyanoacrylates; poly-.beta.-maleic acid (PMLA), polyalkanotes and poly-.beta.-alkanoic acids. There are many other degradable materials known in the art. (See e.g., Biomaterials Science: An Introduction to Materials in Medicine (29 Jul. 2004) Ratner, Hoffman, Schoen, and Lemons; and Atala, A., Mooney, D. Synthetic Biodegradable Polymer Scaffolds. 1997 Birkhauser, Boston; incorporated herein by reference).

Natural polymers (biopolymers) include any protein or peptide. For example but not limited to chitosan and collagen and other polypeptides and proteins, and any combinations thereof. In yet another alternative embodiment, shape-shifting polymers may be used to fabricate stents constructed according to the present invention. Suitable shape-shifting polymers may be selected for instance from the group consisting of polyhydroxy acids and polyorthoesters and copolymers thereof and those of U.S. Pat. Nos. 6,160,084 and 6,388,043 and 6,720,402, each of which are incorporated by reference herein. In some embodiments, the filaments may comprise layers of materials.

Resorbable polymers offer much greater flexibility than metals of any kind for local delivery of “therapeutic agents” (for example, a pharmnaceutical agent and/or a biologic agent) sufficient to exert a selected therapeutic effect. The term “pharmaceutical agent”, as used herein, encompasses a substance intended for mitigation, treatment, or prevention of disease that stimulates a specific physiologic (metabolic) response. The term “biological agent”, as used herein, encompasses any substance that possesses structural and/or functional activity in a biological system, including without limitation, organ, tissue or cell based derivatives, cells, viruses, vectors, nucleic-acids (animal, plant, microbial, and viral) that are natural and recombinant and synthetic in origin and of any sequence and size, antibodies, polynucleotides, oligonucleotides, cDNA's, oncogenes, proteins, peptides, amino acids, lipoproteins, glycoproteins, lipids, carbohydrates, polysaccharides, lipids, liposomes, or other cellular components or organelles for instance receptors and ligands. Further the term “biological agent”, as used herein, includes virus, serum, toxin, antitoxin, vaccine, blood, blood component or derivative, allergenic product, or analogous product, or arsphenamine or its derivatives (or any trivalent organic arsenic compound) applicable to the prevention, treatment, or cure of diseases or injuries of man (per Section 351(a) of the Public Health Service Act (42 U.S.C. 262(a)). Further the term “biological agent” may include 1) “biomolecule”, as used herein, encompassing a biologically active peptide, protein, carbohydrate, vitamin, lipid, or nucleic acid produced by and purified from naturally occurring or recombinant organisms, antibodies, tissues or cell lines or synthetic analogs of such molecules; 2) “genetic material” as used herein, encompassing nucleic acid (either deoxyribonucleic acid (DNA) or ribonucleic acid (RNA), genetic element, gene, factor, allele, operon, structural gene, regulator gene, operator gene, gene complement, genome, genetic code, codon, anticodon, messenger RNA (mRNA), transfer RNA (tRNA), ribosomal extrachromosomal genetic element, plasmagene, plasmid, transposon, gene mutation, gene sequence, exon, intron, and, 3) “processed biologics”, as used herein, such as cells, tissues or organs that have undergone manipulation. The therapeutic agent may also include vitamin or mineral substances or other natural elements.

Through the modification of polymer chemistry these materials can also often be engineered and re-engineered to tailor the body's response with regard to inflammation and toxicity. In contrast to certain biostable polymers and metals, the resorbable polymers generally have lower achievable values of tensile strength and other mechanical properties for load bearing applications. Biostable polymers have the advantage of having better mechanical properties and durability than resorbable polymers.

Biostable metals in general are mechanically robust compared to polymers such that the metal device has a permanent function of taking the load imposed by the tissue or in supporting a tissue. This gives the clinician and patient a high reassurance for device function. Metals offer a major advantage over most polymers in that they are radiopaque. Erodible or corrodible metals, like polymers that degrade, allow the tissue to be under less stress and strain as the metals oxidize and break apart. Yet release of nonresorbable wear particles in tissues can cause undesirable biological responses. Use of these materials would preferably be restricted to body areas where tissues may embed any such particles.

Any of the embodiments can advantageously be coated with swelling hydrogels and/or therapeutic agents which can promote tissue growth or thrombosis to assist the base device to occlude the aneurysm or other cavity. Additionally non-swelling coatings of any composition may be applied to achieve a similar effect. In some embodiments, the filament 12 has a differential cross-section (for example, notched) at various points along their length. In other embodiments, the filament 12 has a substantially constant cross section. As discussed further below, the differential and constant cross section embodiments allow for selection to suit a particular need such as in connection with pushability, flexibility and detachment method of the device.

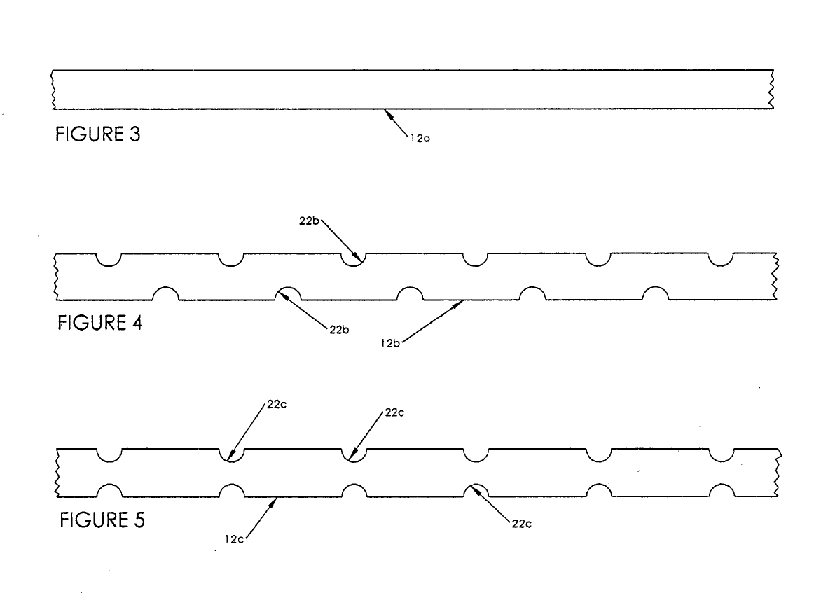

FIG. 3 shows a non-notched embolic filament 12a. The filament 12a has a substantially constant cross-section along its entire length. Preferably, the cross-section of the filament 12a is substantially circular or round though in modified embodiments other suitable shapes may be utilized with efficacy, for example, oval, ellipsoidal and the like. In preferred embodiments, the filament has an outside diameter of about 0.001 to about 0.1 inches, and more preferably, from about 0.003 to about 0.015 inches.

FIG. 4 shows a notched embolic filament 12b. The filament 12b includes a plurality of spaced grooves or notches 22b arranged in a predetermined manner along its length. In the illustrated embodiment, the notches 22b are arranged in a staggered alternating configuration, though other suitable arrangements may be used, as needed or desired. Each of the notches 22b partially circumscribes a portion of the filament outermost periphery.

Preferably, the cross-section of the filament 12b is substantially circular or round, at least at the non-notched portions, though in modified embodiments other suitable shapes may be utilized with efficacy, for example, oval, ellipsoidal and the like. As discussed further below, the notches or grooves 22b preferably aid detachment of the filament 12b from a catheter.

FIG. 5 shows another embodiment of a notched filament 12c in which spaced grooves or notches 22c substantially entirely circumscribe the filament's outermost periphery, that is, preferably extend all the way around. The notches 22c are arranged in a predetermined manner along the length of the filament 12c. In the illustrated embodiment, the notches 22c are arranged substantially equidistantly from adjacent notches though other suitable arrangements may be used, as needed or desired.

Preferably, the cross-section of the filament 12c is substantially circular or round, at least at the non-notched portions, though in modified embodiments other suitable shapes may be utilized with efficacy, for example, oval, ellipsoidal and the like. As discussed further below, the notches or grooves 22c allow for detachment of the filament 12c from a catheter.

### Embolic Filament Advancement

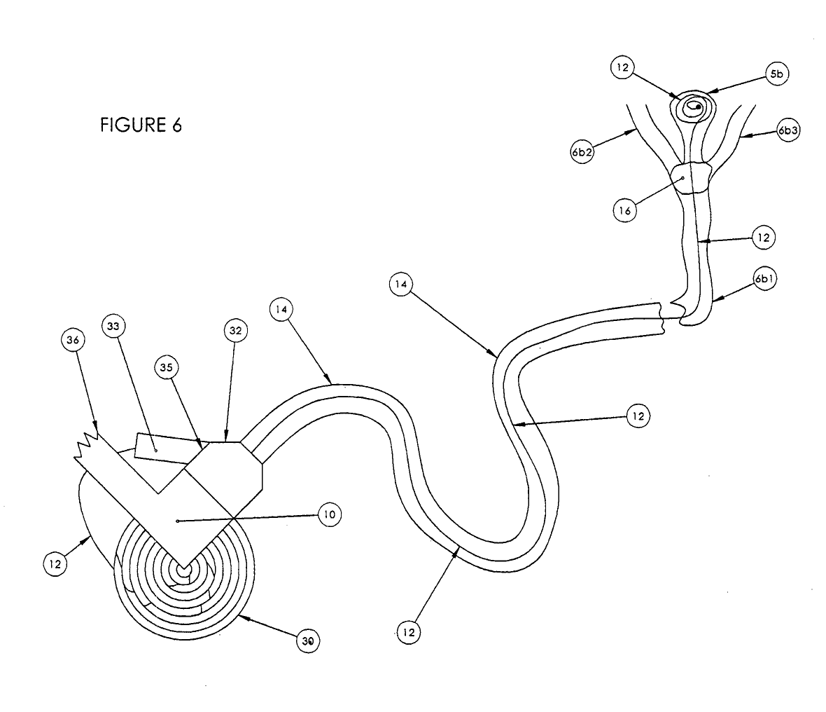

FIG. 6 shows the apparatus or system 10 including an embolic filament spool device or system 30 advancing the embolic filament 12 to the aneurysm 5b through the guiding catheter 14. The filament dispensing device 30 is interfaced with the catheter 14 at a proximal hub luer lock 32 of the catheter 14 and includes a filament spool portion 34 and a loading transfer tube 33 with an interfacing hub luer lock 35.

The drawn filament 12 is stored in the spool device 30 which also keeps the embolic filament 12 sterile. The filament dispensing device 30 includes a filament advancing mechanism 36 which is situated between the filament spool 32 and the guiding catheter 14. This mechanism can have several configurations but generally comprises a series of cam and gear mechanisms to grab and support the thin filament 12 while advancing it distally into the guiding catheter 14.

An advancement lever 38 (e.g., a thumb-wheel) is manually, electromechanically or operatively controlled by a user to advance (or retract) the filament 12 to load it into the delivery catheter 14. As discussed above, the distal end of the filament device 12 has a special pre-formed “starter” blunt end 20 on it to ensure that this end will not puncture or cause rupture of the aneurysm sac 7b. The filament 12 is loaded into the guiding catheter 14 which serves as the internal transport conduit to enable the filament 12 to reach the embolic site.

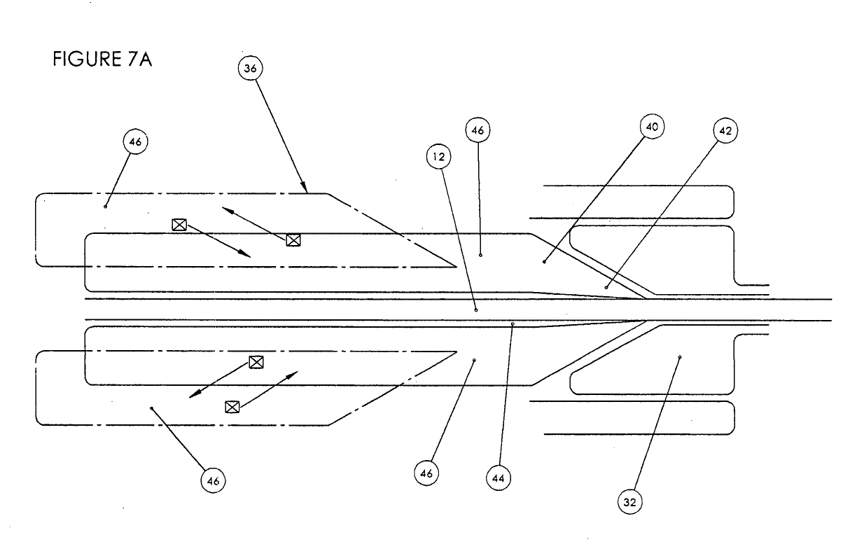

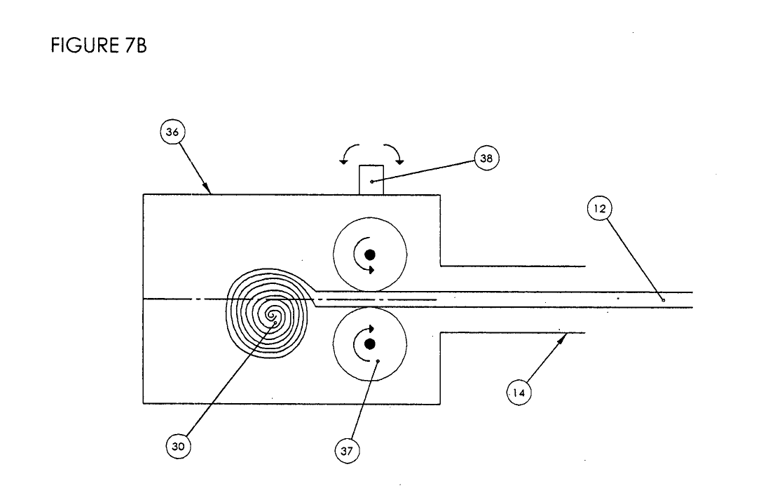

FIG. 7A illustrates the operation of the filament advancement device 36 in accordance with one embodiment. The filament advancement device 36 includes a distal tip 40 with a distal end 42 and a variable size passage 44 extending therethrough for accommodating the embolic filament 12. The filament advancement device 36 may comprise two or more radially and longitudinally displaceable members 46.

The gripping members 46 are shown in the extended “pushing” position and also in phantom in the retracted position. The passage 44 near the distal end 42 tapers inwards so as to engage the filament 12. The distal tip 40 is tapered and abuts against the guiding catheter hub 32 in the fully extended position.

In use, the filament advancement device 36 is operated to grip the filament 12 and advance it longitudinally into the guiding catheter 14 through the catheter hub 32. After the fully extended position is reached, the filament advancement device 36 is retracted. This process is repeated until a desired or suitable length of the filament 12 has been provided to the embolic site.

A preferred alternative embodiment of the spool delivery device is illustrated in FIG. 7B. In this embodiment, the advancement mechanism 36 comprises motorized wheels 37, which are preferably sterile. Operation of the advancement lever 38 switches on an electric motor that drives the wheels. The wheels are made of a material having the physical characteristics adapted to create a frictional engagement with the filament 12. The wheels may be formed of a rubber or other deformable material and are preferably positioned with a gap that is smaller than the diameter of the filament, such that the opposing wheels contact the filament with partial deformation or compression to facilitate positive frictional drive. As illustrated in FIG. 7B, the wheels spin in opposite directions (one clockwise and the other counterclockwise) so the filament can be advanced or retracted. The motor and electronics are configured to allow forward and reverse drive.

### Embolic Filament Detachment

Once the continuous embolic filament 12 has been placed at or within the target site, at least a portion of the length of the embolic material is detached and remains at the intended deposition site. In the embodiments using a polymer as the embolic material, the detachment can be accomplished in many ways including, but not limited to, the embodiments disclosed, taught or suggested herein.

In some embodiments, the embolic filament 12 includes a geometry with a break away joint which couples the implantable embolic section with the delivery section of the filament 12. In some embodiments, the joint supports compression but detaches into two pieces after it is exposed to a particular level of tensile force resulting in the generation of a particular level of tensile stress. As discussed in further detail below, this level of tensile stress can be imparted by hydrostatic fluid pressure when in combination with a guiding catheter design. This design incorporates a fluid injection lumen which fills an internal device guiding lumen with fluid pressure near the exit tip of the guiding catheter.

In other embodiments, the joint of the embolic filament 12 supports compression but detaches into two pieces after it is exposed to a particular level of torsional force resulting in the generation of a particular level of torsional stress. In yet other embodiments, the joint of the embolic filament 12 supports compression but detaches into two pieces after the joint is exposed to a particular level of combined loading (which includes tensile force and torsional force) resulting in the generation of a particular level of combined stress loading, that is, both tensile and torsional or combinations of hydrostatic force and tensile, torsional or compressive stress.

In some embodiments, the filament 12 is synthesized from a resorbable or non-resorbable polymer which has mechanical properties designed to support compressive stress but not to support the same level of tensile stress, thereby allowing fracture at a selected location. In some embodiments, this filament 12 is radiopaque.

In some embodiments, the embolic filament 12 is cut through or fractured using a specially designed guiding catheter. As discussed further below, the guiding catheter has a stress concentrator which is actuated by filling an actuating lumen which runs substantially parallel with the guiding catheter lumen (which contains the embolic filament). Any of the filament detachment embodiments may be efficaciously combined, as needed or desired.

Embodiments of the invention, desirably allow the filament 12 to be reliably detached, often deep, within the vasculature. As discussed above in connection with FIGS. 4 and 5, the filament 12 can have areas of reduced cross sectional area to serve as preferential detachment points. These reduced cross sections, grooves or notches 22 are spaced frequently along the filament longitudinal axis at a predetermined spacing or distance. This allows enablement of an appropriate “detachment length resolution” in order to ensure the aneurysm or cavity is neither under filled nor over filled with the embolic filament 12. The grooves or notches 22b in FIG. 4 may be spaced from about 0.002 to about 1 inch and more preferably from about 0.005 to about 0.25 inches. The double or opposing notches 22c in FIG. 5 may be spaced from about 0.001 to about 0.5 inches and more preferably from about 0.0025 to about 0.125 inches

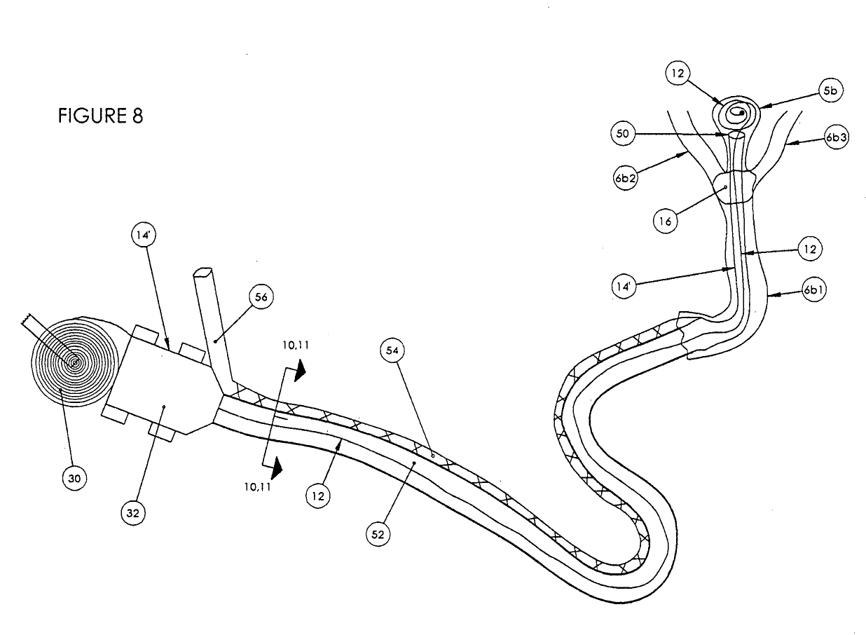

FIG. 8 shows a dual lumen pressurized guiding catheter 14′ for fracturing the notched embolic filament 12 (12b, 12c). As discussed further below, the filament fracturing preferably occurs within the catheter 14′ and proximate a distal tip 50 of the catheter 14′.

The guiding catheter 14′ includes a main lumen 52 that receives the embolic filament 12 advanced by the spool device 30. The guiding catheter 14′ further includes a pressurization lumen 54 that preferably runs substantially the entire length of the catheter 14′. A “detachment” pressurization port 56 is in fluid communication with the pressurization lumen 54 and is located at or proximate to the catheter hub 32. As discussed further below, the port 56 is used to provide fluid to the lumen 54 which provides fluid pressure assistance to fracture the notched embolic filament 12 (12b, 12c).

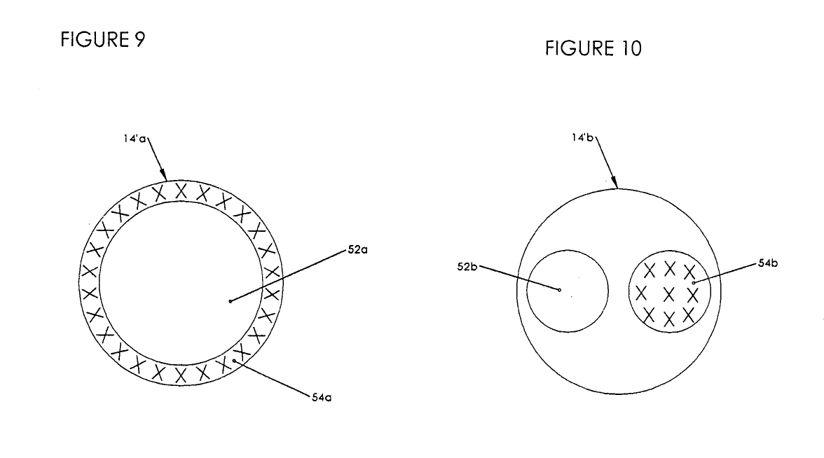

FIG. 9 is a sectional view illustrating the dual lumen arrangement of a catheter 14a′ in accordance with one embodiment. In the illustrated embodiment, the internal filament-receiving lumen 52a is substantially circumscribed or surrounded by the external pressurization lumen 54a that preferably runs substantially the entire length of the catheter 14a′.

FIG. 10 is a sectional view illustrating the dual lumen arrangement of a catheter 14b′ in accordance with another embodiment. In the illustrated embodiment, the internal filament-receiving lumen 52b and the external pressurization lumen 54b are positioned adjacent to one another in a side-by-side configuration. The pressurization lumen 54b preferably runs substantially the entire length of the catheter 14b′.

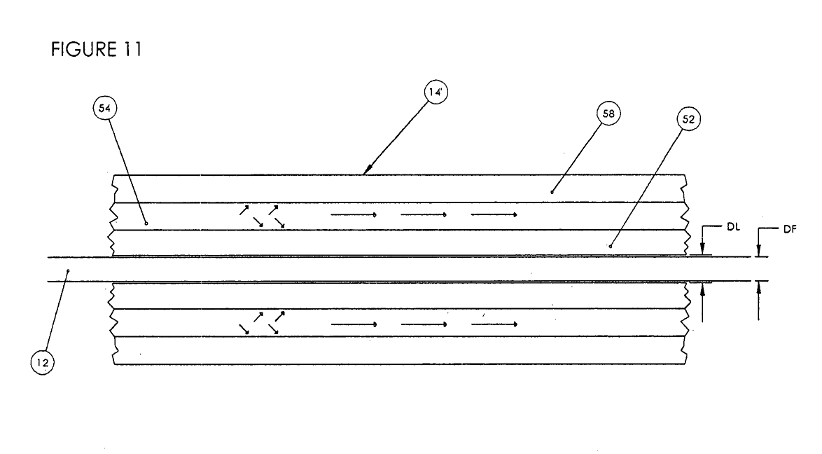

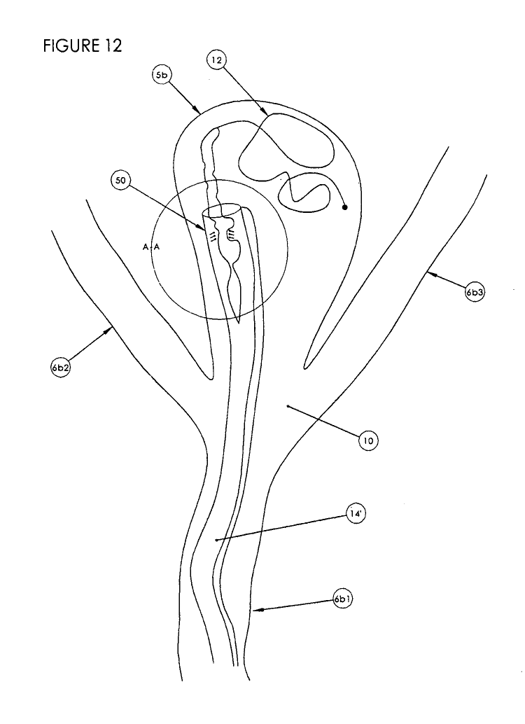

FIG. 11 shows a close-up view of the embolic filament 12 within the internal lumen 52 of the guiding catheter 14′. An important parameter relating to the “pushability” of the embolic filament 12 as it is dispensed from the spool device 30 into the catheter 14′ is the gap clearance between the inner dimension of the catheter 14′ (e.g. the diameter D_{L} of the internal lumen 52) and the outer dimension or diameter D_{F} of the embolic filament 12. Thus, the gap clearance G_{C} is given by:

$${G}_{C} = \frac{{D}_{L} - {D}_{F}}{2}$$

Both the embolic filament 12 and the filament-receiving catheter internal lumen 52 are designed and constructed to tightly controlled tolerances to provide a substantially uniform, though small, gap clearance G_{C} that allows sufficient space for the filament 12 to be moved through the internal lumen 52 while maintaining a generally smooth longitudinal advancement and avoiding undesirable impedance-to the forward motion. In the illustrated embodiment, the guiding catheter 14′ includes an outer braided reinforcement 58. In preferred embodiments, the lumen has an inside diameter of about 0.001 to about 0.050 inches, and more preferably about 0.010 inches. In preferred embodiments, the filament has an outside diameter of about 0.0005 to about 0.0495 inches, and more preferably about 0.009 inches. In preferred embodiments, the gap clearance is about 0.0005 to about 0.0495 inches, and more preferably about 0.003 inches.

FIG. 12 illustrates the process of pressurized detachment of the notched embolic filament 12 (12b, 12c) using the guiding catheter 14′. The detachment occurs at or proximate the distal end 50 of the guiding catheter 14′ once a sufficient amount of filament has been packed in the aneurysm 5b to embolize it. (In FIG. 12, for clarity, only a portion of the embolic filament 12 is shown within the aneurysm 5b.)

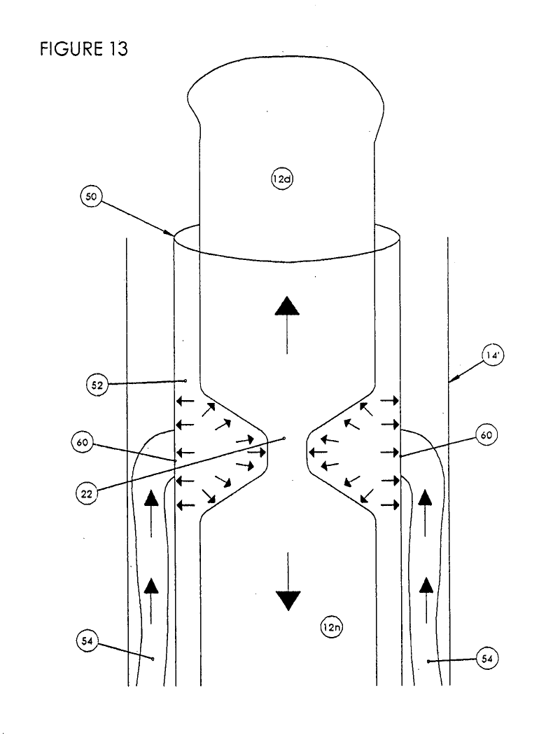

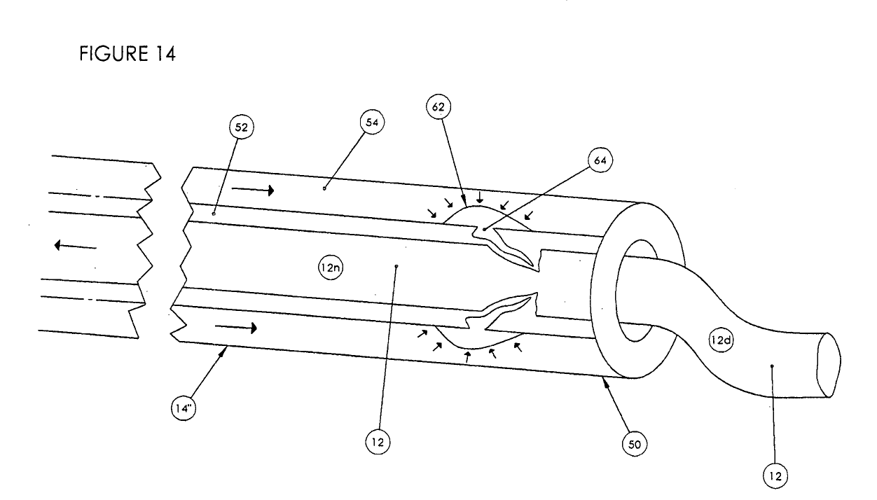

FIG. 13 shows in more detail the process of pressurized detachment of the notched embolic filament 12 (12b, 12c) using the guiding catheter 14′. Though the drawing illustrates the detachment of the double-notched filament 12c (see FIG. 5), the guiding catheter 14′ may efficaciously be utilized in conjunction with the notched filament 12b (see FIG. 4). Other suitable configurations of embolic filaments with preferential reduced cross sections which provide detachment locations are also included in embodiments of the invention.

The guiding catheter 14′ includes one or more fluid introductions lumens or ports 60 that allow fluid communication between the pressurization lumen 54 and the internal lumen 52 at or slightly proximal to the distal tip 50 of the guiding catheter 14′. The fluid introduction lumens or ports 60 assist in detachment at the reduced cross section(s) 22 by applying or inducing a fluid pressure to impart a tensile separation force F_{R} to detach the deployed embolic filament portion 12d from the non-deployed embolic filament 12n. The pressurization fluid is provided through the detachment pressurization port 56 (see FIG. 8). In preferred embodiments, the pressurized fluid is saline or blood, and more preferably saline. The pressure is preferably in the range of about 0.5 to about 3000 psi, and more preferably about 200 psi. Thus, detachment of the embolic filament 12 may be caused for example when the imparted fluid pressure fractures the filament 12 and divides it into the deployed embolic filament portion 12d and the non-deployed embolic filament 12n. The deployed embolic filament portion 12d embolizes the aneurysm 5b while the non-deployed embolic filament 12n is removed from the patient.

FIG. 14 shows a dual lumen cutting and guiding catheter 14″ in accordance with another embodiment. The catheter 14″ is generally similar to the catheter 14′ except that instead of fluid introduction ports or lumens 60 it includes a hydraulically activated embolic filament cutting device 62 with one or more cutters 62. The delivery lumen 52 accommodates the embolic filament 12. The fluid pressure lumen 54 imparts pressure at or slightly proximal to the catheter distal tip 50 to induce filament detachment by the hydraulically actuated stress concentrator 62 placed at or proximate to the distal tip 50.

The delivery lumen 52 may be substantially circumscribed or surrounded by the external pressurization lumen 54 that preferably runs substantially the entire length of the catheter 14″ (as shown in FIG. 14 and discussed above in connection with FIG. 11). In other embodiments, the internal filament-receiving lumen 52 and the pressurization lumen 54 are positioned adjacent to one another in a side-by-side configuration (as discussed above in connection with FIG. 10).

The cutters 64 are displaced radially inward in response to pressure applied through the fluid lumen and fracture the filament 12 and divide it into the deployed embolic filament portion 12d and the non-deployed embolic filament 12n. The deployed embolic filament portion 12d embolizes the aneurysm 5b while the non-deployed embolic filament 12n is removed from the patient. In preferred embodiments, the pressurized fluid is saline or blood, and more preferably saline. The pressure is preferably in the range of about 0.5 to about 3000 psi, and more preferably about 200 psi.

The cutting-guiding catheter 14″ has particular efficacy for use in conjunction with non-notched filaments 12 (12a in FIG. 3). In modified embodiments, the cutting-guiding catheter 14″ may be used with notched filaments 12 (12b, 12c), as needed or desired.

### Method of Embolizing a Neurovascular Aneurysm with an Embolic Filament

The approximate or exact volume of the cavity to be embolized is determined. This can be done in a number of ways including, but not limited to, quantitative coronary angiography (QCA), magnetic resonance imaging (MRI), contrast assisted MRI, X-ray, among others.

A first neurological guide wire is installed into the aneurysm cavity. A second neurological guide wire is installed either inside the aneurysm or longitudinally across and distal to the aneurysm neck.

A neurovascular guiding catheter is tracked along the first wire into the aneurysm sac. The guiding catheter can include any of the embodiments of the catheter 14 described and illustrated herein.

A low durometer compliant polymer balloon is tracked into position to bridge the aneurysm neck (see, for example, the balloon 16 illustrated in FIG. 2). The balloon is inflated to gently bridge and seal the aneurysm neck and pin the delivery catheter against the side of the neck. This is tested with contrast flow through the guiding catheter to ensure that the aneurysm neck is sealed with balloon pressure sufficient just to allow small amounts (wisps) of contrast agent to seep from the balloon-aneurysm neck interface. The first neurological guide wire is removed while the balloon is inflated.

The embolic filament is loaded into the delivery catheter by first connecting, if not already connected, the hub luer lock of the loading transfer tube to the hub luer lock of the micro guiding catheter. A “pushing force” is introduced to push or advance the embolic device within and through the guiding catheter. This force may be applied in a number of manners and some embodiments of which are described herein and above.

In some embodiments, the embolic filament spool device 30 (see, for example, FIGS. 6-8) is used to advance the embolic filament to the aneurysm site. In modified embodiments, other suitable pushing mechanisms such as fluid pressure and/or a mechanical pushing device member can be used to advance the embolic filament.

The advancement and positioning of the embolic device within the delivery catheter and into the aneurysm site is monitored using visualization techniques. These include, but not limited to, QCA, MRI, contrast assisted MRI, X-ray, among others.

The embolic filament is continued to be fed through the catheter until the desired packing density is achieved inside the aneurysm or other body cavity. As the embolic filament displaces the contrast material from the aneurysm sac, the contrast fluid seeps out around the balloon-aneurysm neck interface. This is confirmed by performing QCA digital subtraction or other suitable visualization techniques.

Once the desired results have been confirmed, the embolic filament is detached. Any one of the embodiments described and illustrated herein and above can be used to detach the filament.

After embolization of the aneurysm, the pressure within the inflated balloon is slowly released while ensuring that the embolic device(s) are stable. The balloon and the second guide wire are removed from the patient to substantially complete the embolization of the neurovascular embolism. During the procedure, any of the visualization techniques and equipment as taught or suggested herein may be used to view the progress during the procedure, as needed or desired.

### Bundled Embolic Filament Embodiment

Some embodiments relate to a plurality of filament structures which are bundled together to occlude aneurysms in the neurovasculature or other sites where embolization is required to satisfy a particular clinical objective. These filaments preferably have a slenderness ratio (length to width ratio) which individually provides minimal bending stiffness in order not to perforate the tissue of the site to be embolized.

For example, the stiffness of a single filament individually may not be strong enough to be pushed through a delivery catheter nor radiopaque enough to be seen fluoroscopically. But, when a plurality of these filaments are collectively bundled, they become structural or stiff enough in nature to be pushed to the treatment site and radiopaque due to their collective geometry and mass.

These filaments may be bundled together at any suitable position along their length, as discussed further below, in order to provide a variety of enhanced functions for embolizing and occluding a body cavity. These functions include, but are not limited to, bundling to increase the pushability of the embolic device through the delivery catheter, bundling to increase the displacement volume of the embolic device and bundling to enhance radiopacity.

Advantageously, these bundled embolic filaments may be deployed at the target site by a pushing device without a detaching or fracturing process. In one embodiment, the pushing device comprises pressurized liquid acting on the projected cross sectional area of the bundled embolic device while it is inside the internal diameter (ID) of a delivery catheter. In another embodiment, the bundled embolic device may be pushed with a mechanical pushing rod and its motion to the embolic site monitored. In still another embodiment, a combination of mechanical pushing with pressure assistance may be employed to advance the bundled filament device to the target site.

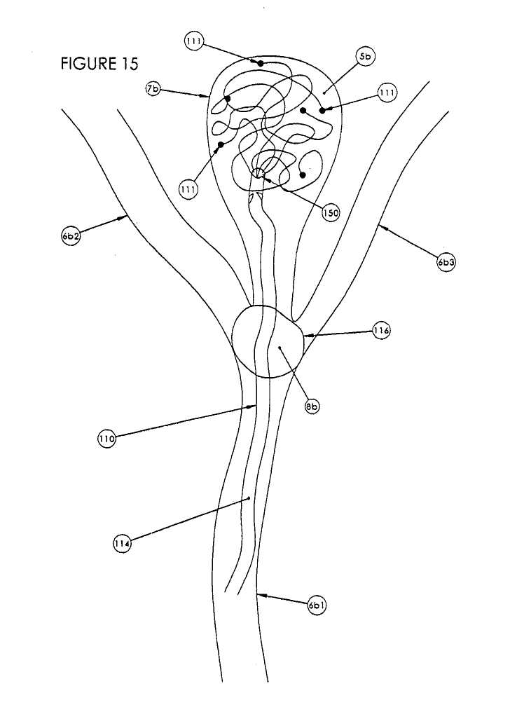

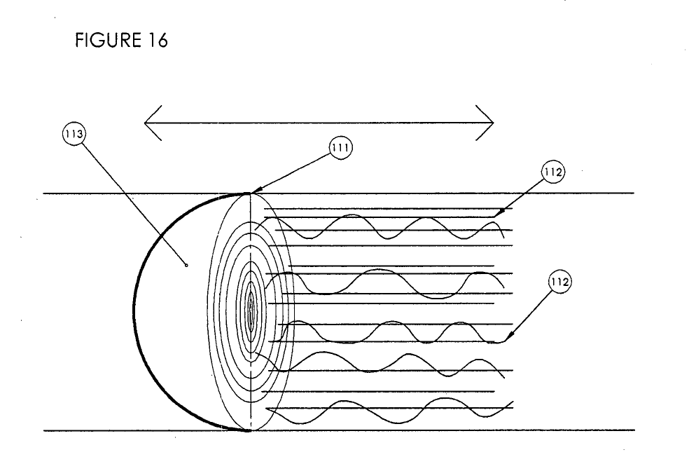

FIG. 15 shows a partial view of an apparatus or system 110 including one or more bundled embolic filament prostheses or devices 111 deployed in the aneurysm 5b utilizing a guiding catheter 114. The prostheses 111 are dispensed from the catheter 114 at an opening at or proximate its distal end 150. As discussed further below, a preferably low durometer compliant balloon 116 is used to bridge the aneurysm neck 8b. In embodiments of the invention, one or more of the bundled embolic filament prostheses 111 can be used to densely pack the aneurysm 5b or other body or luminal cavity to occlude or embolize it.

FIG. 16 shows the bundled embolic filament prosthesis 111 in more detail. The bundled embolic prosthesis 111 generally comprises a plurality of embolic filaments 112 that are bunched at a predetermined position along their length to form a bundled section 113. The top of this prosthesis may have a hemispherically shaped head to prevent perforation of the aneurysm once placed. Advantageously, the bundled section 113 allows for composite stiffness for pushability. Alternatively the prosthesis may be pushed from either direction. In the illustrated embodiment, the bundled section 113 is generally circular.

In one embodiment, the mono filaments 112 have a variable length. In another embodiment, the mono filaments 112 have substantially the same length. In another preferred embodiment, the variable length filaments provide improved packing within the aneurysm. Likewise, variable diameter filaments may provide advantageous functionality in some embodiments. In some embodiments, the filaments 112 within the bundle may be tapered. The bundled filament prostheses illustrated in FIG. 16 may be pushed in either direction, e.g., with the bundled section 113 disposed distally (in the direction of the advancement) or in other embodiments, the bundled section 113 may be disposed proximally with respect to the direction of advancement. The distal orientation is preferred in some embodiments because the hemispherically shaped bundle section 113 may prevent perforation of the aneurysm. Preferably, the cross-section of the filaments 112 is substantially circular or round, though in modified embodiments other suitable shapes may be utilized with efficacy, for example, oval, ellipsoidal and the like.

The prosthesis 111 can be fabricated by any one of a number of manufacturing techniques. For example when using metal, the filaments 112 can be made by a hot or cold drawing process. In the case of polymer filaments, the filaments 112 can be made by an extrusion process and secondary hot or cold drawing process. The filaments 112 are bonded to form the bundled section 113 using heat bonding or with a nontoxic thrombotic adhesive.

In some embodiments, the filaments 112 comprise radiopaque or non-radiopaque polymers. In some embodiments, the filaments 112 comprise biodegradable, degradable or non-resorbable polymers. In some embodiments, the filaments 112 comprise erodible or non-erodible metals. In some embodiments, the filaments 112 comprise shape memory metals such as, but not limited to, Nitinol and spring steel. Any combination of these embodiments may be efficaciously utilized, as needed or desired. Any of the embodiments can advantageously be coated with polymers (e.g., swelling hydrogels) and/or therapeutic agents (e.g., pharmaceutical compounds or proteins or genetic materials) which can promote a desired tissue response (e.g., tissue growth or thrombosis) to assist the base device to occlude the aneurysm or other cavity.

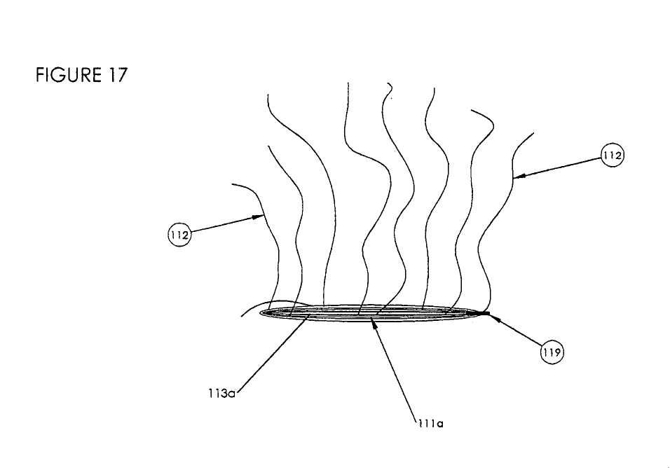

FIG. 17 shows one embodiment of a bundled multi-filament embolic device or prosthesis 111a. The bundled embolic device 111a comprises a bundled joint section 113a with bonded filaments 112 at a proximal end 119 of the device 111a.

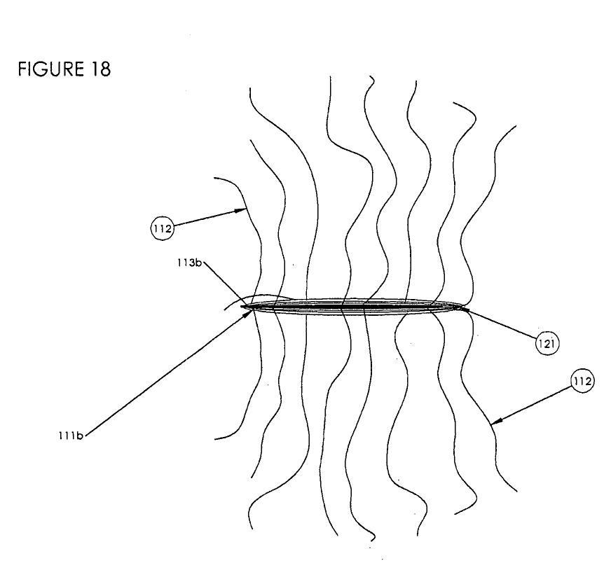

FIG. 18 shows another embodiment of a bundled multi-filament embolic device or prosthesis 111b. The bundled embolic device 111b comprises a bundled joint section 113b with bonded filaments 112 at substantially a middle section 121 of the device 111b.

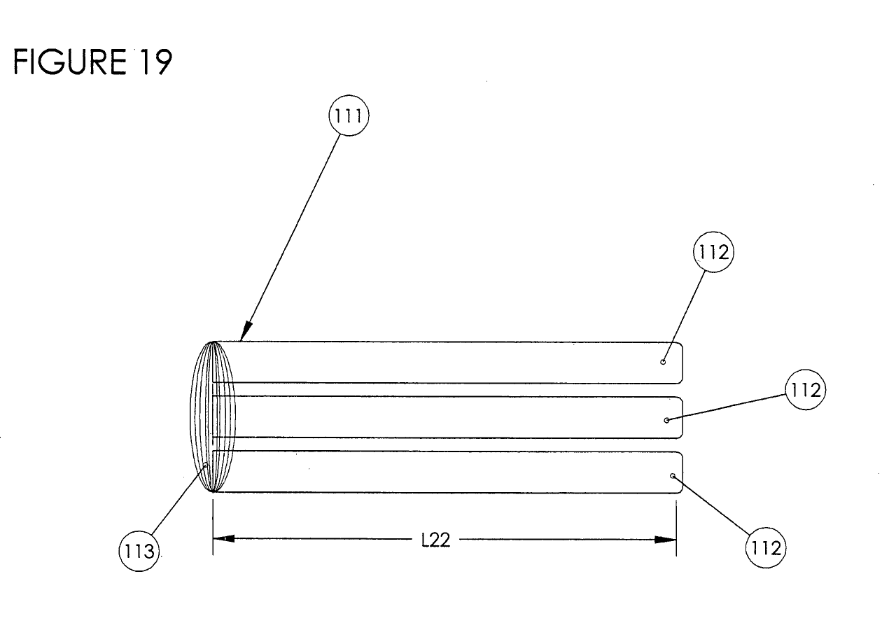

FIG. 19 shows the bundled embolic filament prosthesis 112 (112a) with the longitudinal (non-coiled) mono filaments in an extended or generally straight arrangement. In preferred embodiments, the overall prosthesis length L₂₂ may range from about 0.005 to about 2.000 inches, more preferably, L₂₂ is about 0.060 inches.

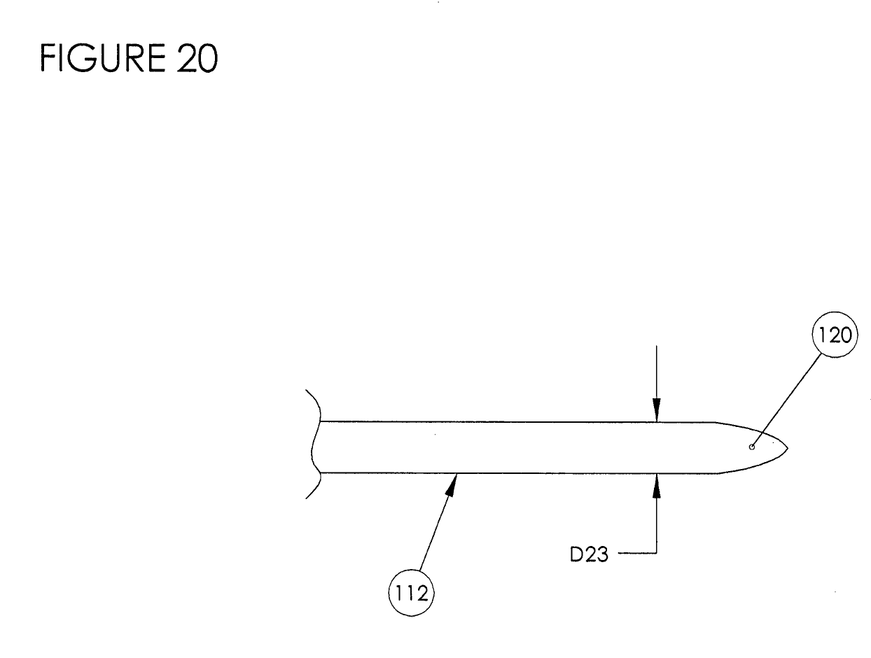

FIG. 20 shows a distal end or tip 120 of one of the filaments 112. In the embodiment of FIG. 20, the distal end is generally tapered while the remaining portion of the filament 112 has substantially uniform dimension or diameter D₂₃. In one embodiment, the diameter D₂₃ is about 12.7±3.81 microns or μm (0.0005±0.00015 inches).

Since the filaments 112 preferably have a slenderness ratio (length to width ratio) which individually provides minimal bending stiffness, the distal tips 120 do not perforate the tissue of the site to be embolized. In modified embodiments, the filament distal tips may include a blunt or rounded end, as needed or desired. In one embodiment, the mono filaments 112 have a variable length. In another embodiment, the mono filaments 112 have substantially the same length. In another preferred embodiment, the variable length filaments provide improved packing within the aneurysm. Likewise, variable diameter filaments may provide advantageous functionality in some embodiments. In some embodiments, the filaments 112 within the bundle may be tapered. The bundled filament prostheses illustrated in FIG. 16 may be pushed in either direction, e.g., with the bundled section 113 disposed distally (in the direction of the advancement) or in other embodiments, the bundled section 113 may be disposed proximally with respect to the direction of advancement. The distal orientation is preferred in some embodiments because the hemispherically shaped bundle section 113 may prevent perforation of the aneurysm. Preferably, the cross-section of the filaments 112 is substantially circular or round, though in modified embodiments other suitable shapes may be utilized with efficacy, for example, oval, ellipsoidal and the like.

As discussed above with respect to FIGS. 4 and 5, a filament 12 with a differential cross-section (for example, notched) at various points along its length. A plurality of notches or grooves 22 may be spaced at predetermined locations along the filament length. The notches or grooves 22 can also extend substantially fully around the circumferential periphery of the filament 12. The differential cross section embodiments allow for selection to suit a particular need such as in connection with flexibility without rupturing the aneurysm, pushability and packing efficiency of the device within the aneurysm. In one embodiment, the diameter D₂₄ is about 20.3 microns or μm (0.0008 inches) and the notch depth H₂₄ is about 5.1 μm (0.0002 inches).

### Bundled Embolic Filament Advancement

The bundled embolic filament may be advanced using conventional pushing rods that include a generally elongated pusher tube, shaft, shank or stem mechanically connected to a handle at its proximal end. Of course, any pushing device known in the art, with any configuration adapted to advance the bundled embolic device may be employed in embodiments of the inventive method. Typical pushing rods have a distal end that engages the bundled embolic device(s) to push them through the guiding catheter to the aneurysm site. The shank of the pushing rod is preferably flexible so that it can bend and curve along with the guiding catheter within the blood vessels.

In one preferred embodiment, the handle of the pushing rod is adapted to be operably engaged by a user such as a surgeon. Accordingly, the handle is preferably shaped and contoured to be generally circular or other suitable ergonomic shape that facilitates in the operation of the pushing rod. Alternatively, the pushing rod can be advanced automatically, similar to the auto-feed mechanism shown in FIG. 7.

The pushing rod may be manually, electromechanically or operatively controlled by a user to push and advance the bundled embolic filament prosthesis to load it into the delivery catheter. The delivery lumen of the guiding catheter may serve as the internal transport conduit to enable the bundled embolic filament prosthesis to reach the embolic site.

As discussed further below, more than one bundled embolic filament prosthesis may be loaded into the advancement mechanism and or guiding catheter and simultaneously advanced to the embolic site. In some embodiments, single bundled embolic filament prostheses are sequentially advanced to the embolic site, that is, the advancement device, e.g., the pushing rod, is retracted after placement of the single prosthesis in the aneurysm and another individual prosthesis loaded and advanced to the embolic site. This is repeated until the desired or suitable number of embolic prostheses have been delivered to densely pack the aneurysm and embolize it.

A combination of simultaneous and sequential prosthesis delivery may also be used with efficacy, as needed or desired. For example, twelve embolic prostheses may be delivered to the embolic site in groups of three or four and the like.

Advantageously, the bundled embolic filaments are deployed at the target site by a pushing device without a detaching or fracturing process. In one embodiment, the pushing device comprises pressurized liquid acting on the projected cross sectional area of the bundled embolic device while it is inside the internal diameter (ID) of the transfer tube and/or the delivery catheter. In another embodiment, a combination of mechanical pushing (e.g. using a pushing rod) in combination with fluid pressure assistance may be employed to advance the bundled filament device to the target site. For example, the pushing rod may have a lumen therethrough which serves as a conduit for pressurized fluid to advance the embolic device both mechanically via the rod's pushing force and hydraulically using the liquid pressurizing force.

### Multiple Bundles of Embolic Filament

In a variation, a plurality of the bundled embolic filament prostheses may be placed in the delivery lumen of the guiding catheter. The bundled embolic filament prostheses may be arranged, for example, generally longitudinally and serially within the catheter lumen. As discussed above, a pushing mechanism is utilized to deliver and place the desired or suitable number of prostheses 111 at the embolic site.

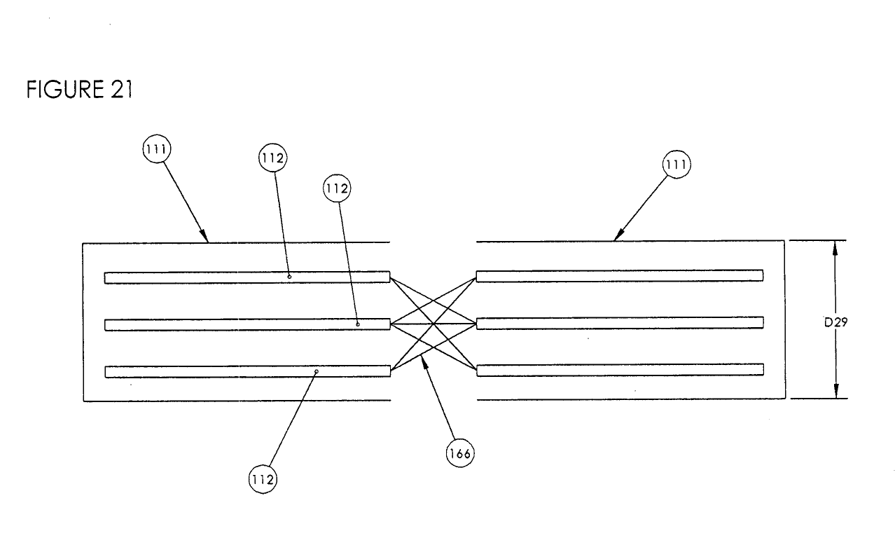

FIG. 21 shows two bundled embolic prostheses 111 that are serially connected to one another to facilitate their advancement and delivery to the embolic site. The filaments 112 of these bundled prostheses 111 are connected by thread elements 166.

Referring in particular to FIG. 21, in one embodiment, the diameter D₂₉ of the embolic device 111 is about 0.38 mm (0.015 inches). In modified embodiments, other suitable diameters may be utilized with efficacy, as needed or desired, depending on the particular use and application.

### Method of Embolizing a Neurovascular Aneurysm with a Bundled Embolic Filament

The approximate or exact volume of the cavity to be embolized is determined. This can be done in a number of ways including, but not limited to, quantitative coronary angiography (QCA), magnetic resonance imaging (MRI), contrast assisted MRI, X-ray, among others

A first neurological guide wire is installed into the aneurysm cavity. A second neurological guide wire is installed either inside the aneurysm or longitudinally across and distal to the aneurysm neck.

A neurovascular guiding catheter is tracked along the first wire into the aneurysm sac. The guiding catheter can include any of the embodiments of the catheter 114 described and illustrated herein.

A low durometer compliant polymer balloon is tracked into position to bridge the aneurysm neck (see, for example, the balloon 116 illustrated in FIG. 15). The balloon is inflated to gently bridge and seal the aneurysm neck and pin the delivery catheter against the side of the neck to prevent movement. This is tested with contrast flow through the guiding catheter to ensure that the aneurysm neck is sealed with balloon pressure sufficient just to allow small amounts (wisps) of contrast agent to seep from the balloon-aneurysm neck interface. The first neurological guide wire is removed while the balloon is inflated.

The appropriate size of the bundled embolic device and the approximate number of bundled embolic devices are selected based on the size of the aneurysm that is to be densely packed and embolized.

The bundled embolic device is loaded into the delivery catheter by first connecting, if not already connected, the hub luer lock of the loading transfer tube to the hub luer lock of the micro guiding catheter. A “pushing force” is introduced to push or advance the embolic device within and through the guiding catheter. This force may be applied in a number of manners and some embodiments of which are described herein and above.

In some embodiments, the embolic advancement device including the mechanical pushing rod is used to advance the bundled embolic device to the aneurysm site. In modified embodiments, other suitable pushing mechanisms such as fluid pressure and/or other mechanical pushing device members can be used to advance the bundled embolic device. In other embodiments, a combination of mechanical pushing force and liquid pressure may be utilized, as needed or desired.

The advancement and positioning of the embolic device within the delivery catheter and into the aneurysm site is monitored using visualization techniques. These include, but not limited to, QCA, MRI, contrast assisted MRI, X-ray, among others.

The embolic filament is continued to be fed through the catheter until the desired packing density is achieved inside the aneurysm or other body cavity. As the embolic filament displaces the contrast material from the aneurysm sac, the contrast fluid seeps out around the balloon-aneurysm neck interface. This is confirmed by performing QCA digital subtraction or other suitable visualization techniques.

The bundled embolic prosthesis is pushed until it is inside the aneurysm. Note contrast fluid will be displaced due to seepage around the balloon-neck interface. The procedure is repeated with additional bundled embolic devices until the aneurysm is filled to prevent neck recannalization.

As noted above, more than one or all of the bundled embolic devices may be introduced into the catheter one behind the other, advanced and packed in the aneurysm substantially simultaneously with the pushing force. This can advantageously reduce the time of the surgery.

The embolization is confirmed by performing QCA digital subtraction or other suitable visualization techniques.

After embolization of the aneurysm, the pressure within the inflated balloon is slowly released while ensuring that the embolic device(s) are stable. The balloon and the second guide wire are removed from the patient to substantially complete the embolization of the neurovascular embolism. During the procedure, any of the visualization techniques and equipment as taught or suggested herein may be used to view the progress during the procedure, as needed or desired.

From the foregoing description, it will be appreciated that a novel approach for forming occlusions has been disclosed. While the components, techniques and aspects of the invention have been described with a certain degree of particularity, it is manifest that many changes may be made in the specific designs, constructions and methodology herein above described without departing from the spirit and scope of this disclosure.

Various modifications and applications of the invention may occur to those who are skilled in the art, without departing from the true spirit or scope of the invention. It should be understood that the invention is not limited to the embodiments set forth herein for purposes of exemplification, but is to be defined only by a fair reading of the appended claims, including the full range of equivalency to which each element thereof is entitled.

What is claimed is:

1. An embolic filament, comprising a bioresorbable radiopaque material, wherein said filament is configured for occluding a lumen or cavity in need thereof.

2. The embolic filament of claim 1, wherein said material comprises a polymer.

3. The embolic filament of claim 1, wherein said material comprises a radiopaque polymer.

4. The embolic filament of claim 3, wherein said radiopaque polymer is selected from formulae I-XV.

5. The embolic filament of claim 3, wherein said material comprises an erodible or corrodible metal.

6. The embolic filament of claim 1, further comprising notches configured to facilitate detachment of the filament.

7. A device for deploying an embolic filament to an aneurysm, comprising a guiding catheter with a lumen adapted for endoluminal catheterization of the aneurysm; a spooling mechanism comprising a length of the embolic filament of claim 1 wound around a spool; a filament advancing mechanism adapted to advance the filament distally through the guiding catheter; and a filament detachment mechanism adapted to sever the advancing filament thereby facilitating filament deployment within the aneurysm.

8. The device of claim 7, further comprising a compliant balloon configured to bridge the aneurysm neck.

9. A method for embolizing a vascular aneurysm, comprising providing the device of claim 7; catheterizing the aneurysm; engaging the filament advancing mechanism; and engaging the filament detachment mechanism.

10. An embolic filament bundle for occluding an aneurysm, comprising a plurality of embolic filaments and a bundled section where the filaments are bundled together at a predetermined location.

11. The embolic filament bundle of claim 10, wherein the bundled section is shaped to facilitate deployment without causing perforation of the aneurysm.

12. A device for deploying the embolic filament bundle of claim 10 to an aneurysm, comprising a guiding catheter with a lumen adapted for endoluminal catheterization of the aneurysm; and a pushing means for advancing the embolic filament bundle distally through the guiding catheter thereby facilitating embolic filament bundle deployment within the aneurysm.

13. A method for embolizing a vascular aneurysm, comprising providing the device of claim 12; catheterizing the aneurysm; loading at least one embolic filament bundle into the device; and advancing the pushing means thereby deploying the embolic filament bundle.

14. The method of claim 13, wherein the pushing means is a pushing rod.
